(19) United States
(12) Patent Application Publication
(10) Pub. No.: US 2013/0273561 A1
(43) Pub. Date: Oct. 17, 2013

(54) LIPID ENCAPSULATION OF SURFACE ENHANCED RAMAN SCATTERING (SERS) NANOPARTICLES

(71) Applicants: Gilbert C. Walker, Mississauga (CA); Christina M. MacLaughlin, Toronto (CA); Shell IP, Richmond Hill (CA)

(72) Inventors: Gilbert C. Walker, Mississauga (CA); Christina M. MacLaughlin, Toronto (CA); Shell IP, Richmond Hill (CA)

(73) Assignee: THE GOVERNING COUNCIL OF THE UNIVERSITY OF TORONTO, Toronto, ON (CA)

(21) Appl. No.: 13/882,371

(22) PCT Filed: Oct. 31, 2011
(86) PCT No.: PCT/CA11/50683

§ 371 Date: Jul. 2, 2013

Related U.S. Application Data
(60) Provisional application No. 61/408,294, filed on Oct. 29, 2010.

(51) Int. Cl. G01N 33/53

(52) U.S. Cl. CPC G01N 33/53
USPC 435/7.2; 436/501; 264/4.7

(57) ABSTRACT

Phospholipid-microvesicle-encapsulated surface enhanced Raman scattering (SERS) nanoparticles and methods for making the encapsulated particles are described. The encapsulated particles can be used in nanomedicine. Four Raman-active species were used. A bilayer was observed by TEM, and the SERS spectrum of each dye species (SERS reporter) was confirmed.

The invention relates to methods for functionalizing Surface Enhanced Raman Scattering (SERS) metal, including gold nanoparticles by phospholipid encapsulation providing a means of controlling chemical, optical, and targeting properties of the particles. The encapsulated SERS nanoparticles can be conjugated to monoclonal antibodies and other targeting ligands to selectively target cell surface receptors and other analytes.

### BACKGROUND OF THE INVENTION

Metal nanoparticles have been widely studied as promising imaging and therapeutic agents in medical diagnostics^{1,2}. In addition to the plasmonic properties of the particles themselves, these applications require three basic functional elements: (1) a layer to control surface chemistry for biocompatibility, biodistribution, and/or colloidal stability³ (2) a targeting moiety⁴⁻⁶; and (3) Raman-active molecules⁷⁻¹⁰ in the case of surface enhanced Raman scattering (SERS) nanoparticles. There are many methods of modifying the surface of nanoparticles to include these elements that involve both chemisorbed and physisorbed molecules or polymers and combinations therein. Alternatively, liposomes designed for drug-deliveryl^{11,12} often share the first two functional requirements, and various methods of engineering their stability and targeting properties have been widely investigated in literature¹³. In some cases the first requirement is provided by the properties of the vesicle itself¹⁴, while the second may come from peptides, antibodies, glycans, or folate covalently bound to lipid anchors^{12,15,16}. Many of the strategies to address these functions for liposome-drug-delivery^{17,18} can potentially be applied to lipid-coated metal nanoparticles for diagnostics and/or therapeutics, provided that a Raman reporter can be incorporated.

In the literature, encapsulation of inorganic nanoparticles in lipoproteins has been adopted for the purposes of colloidal stability, biocompatibility, and the potential of biological targeting^{19,20}. Also, mixtures of phospholipids and surfactants have been employed as capping agents in the synthesis of metal nanoparticle pearl-necklace structures²¹, and nanowires²². United States Patent Publication No. 2011/015186, published Jun. 23, 2011, describes Polymer encapsulated particles as SERS probes, and a report of Raman active phosholipid gold nanoparticles has recently appeared in the scientific literature (Tam, N. C. N.; Scott, B. M. T.; Voicu, D.; Wilson, B. C.; Zheng, G. Facile synthesis of Raman active phopholipid gold nanoparticles. Bioconjugate Chem. 2010, 21, 2178-2182).

### SUMMARY OF THE INVENTION

The present invention provides methods for functionalizing Surface Enhanced Raman Scattering (SERS) metal, preferably gold, nanoparticles by lipid encapsulation providing a means of controlling chemical, optical, and targeting properties of the particles. In preferred embodiments, the encapsulation layer or layers includes one or more phospholipids as a major component.

An embodiment of the invention provides a method of functionalizing surface enhanced Raman scattering (SERS) gold nanoparticles by phospholipid encapsulation, comprising the steps of;

a) mixing gold nanoparticles with a solution containing organic dye molecules and a suspension of phospholipids to form a mixture;

b) sonicating the mixture to induce phospholipid encapsulation of the gold nanoparticles and organic dye molecules; and

c) separating phospholipid encapsulated gold nanoparticles from unbound phospholipids and organic dye molecules.

Step a) may include first mixing the gold nanoparticles with the organic dye molecules and stirring to facilitate adsorption of the organic dye molecules on an outer surface of the gold nanoparticles, thereafter mixing the gold nanoparticles with organic dye molecules adsorbed thereto with the phospholipids to form said mixture.

Alternatively, step a) may include optionally mixing phospholipids having organic dye molecules covalently bound thereto with other phospholipids, and thereafter mixing the gold nanoparticles with the phospholipids having said organic dye molecules bound thereto to form said mixture.

Alternatively step a) may include first dissolving the organic dye molecules into the suspension of phospholipids thereafter mixing therewith the gold nanoparticles to form the mixture.

Another embodiment of the invention is a method of preparing metal nanoparticles for use in surface enhanced Raman scattering (SERS) in which the method includes:
(a) mixing metal nanoparticles, at least one SERS reporter with an aqueous solution of phospholipids to form a mixture; and
(b) agitating the mixture to induce encapsulation of the nanoparticles and

SERS reporter by a phospholipid layer the phospholipid. Another embodiment of the invention is a method of functionalizing surface enhanced Raman scattering (SERS) gold nanoparticles by encapsulation that includes steps of:

a) mixing gold nanoparticles with a solution containing organic dye molecules and a suspension of phospholipid alone, sphingolipid alone, phospholipid and sphingolipid, phospholipid and sterol, sphingolipid and sterol, or phospholipid and sphingolipid and sterol, wherein said phospholipid is one type or a mixture of phospholipids, said sphingolipid is one type or a mixture of sphingolipids, and said sterol is one type or a mixture of sterols, to form a mixture;

b) sonicating the mixture to induce encapsulation of the gold nanoparticles and organic dye molecules by the phospholipid alone, sphingolipid alone, phospholipid and sphingolipid, phospholipid and sterol, or sphingolipid and sterol, phospholipid and sphingolipid and sterol; and

c) separating encapsulated gold nanoparticles from unbound phospholipid alone, sphingolipid alone, phospholipid and sphingolipid, phospholipid and sterol, or sphingolipid and sterol, phospholipid and sphingolipid and sterol, and organic dye molecules.

A particular embodiment is one in which a ligand is covalently linked to the encapsulating layer of a SERS complex. The ligand is, for example, an antibody or a functionally similar type of molecule that can bind to a partner in a selective manner. In this way a complex can be used to determine if the partner is present or absent, or its location can be determined, or possibly the amount or concentration of the partner. The target partner might be in an analytical sample, or an in vivo determination might be made.

In a particular in vivo embodiment, the ligand is a monoclonal antibody and the target is a cell surface receptor to which the antibody selectively binds.

SERS complexes of the invention are amenable to use in multiplexing applications.

The invention includes a method of determining the status of a cell in a sample. The method includes obtaining the sample from a patient, and contacting the sample with a SERS complex of the invention having a ligand which is an antibody capable of preferentially binding a cell surface receptor (antigen) that is potentially present in the sample. The method can further include determining the quantity of the antigen and/or determining the localization of the antigen. The sample can be obtained from a particular tissue e.g., breast tissue from a patient thought or known to be suffering from breast cancer, or stomach, lung, cortical, or vascular tissue.

In another embodiment, a SERS complex of the invention can be used in predicting an increased likelihood of developing cancer progression by detecting the expression of one or more biomarker in a sample from a subject with bioassays. The biomarker is a target of the ligand of the complex. The method could include, for example, comparing the pattern of biomarker expression to a reference biomarker expression pattern from normal tissue. Such a method could thus be used to monitor the effectiveness of a cancer treatment.

In embodiments in which a SERS complex is biocompatible, such a method could be carried out in vivo i.e., without the need for obtaining a sample from a patient.

A further understanding of the functional and advantageous aspects of the invention can be realized by reference to the following detailed description and drawings.

### BRIEF DESCRIPTION OF THE DRAWINGS

The invention will now be described, by way of non-limiting examples only, reference being made to the accompanying drawings, in which:

FIG. 1 shows a schematic illustration of three methods of incorporating dye molecules to produce lipid-encapsulated gold SERS particles. The solid circle represents the gold nanoparticle, the rings represent lipid bilayer vesicles, and the hexagons represent small dye molecules.

FIG. 2 shows the characterization of Malachite Green isothiocyanate (MGITC) lipid-encapsulated SERS nanoparticles produced using Method 1, in which;

A) shows the chemical structure of MGITC,

B) shows TEM image of a bilayer-encapsulated nanoparticle. The ring around the particle is the lipid bilayer, which measures 4.8 nm averaged from 5 measurements of 5 isolated particles.

C) shows DLS histograms of hydrodynamic radius for stock citrate-coated particles (hatched, grey) and MGITC-lipid-coated SERS nanoparticles (outline, black). Log-normal centroids report average Rₕ=29.8±0.1 nm and 34.1±0.5 nm for stock particles and MGITC-SERS particles respectively;

D) shows LSPR absorption in UV-Vis spectrum of stock (grey) and MGITC-lipid-coated particles (black). LSPR absorption peaks at 534 nm for stock particles and 538 nm for MGITC-lipid-coated particles; and

E) shows SERS spectrum of MGITC-lipid-coated nanoparticles showing strong SERS spectrum recognizable as that of MGITC.

FIG. 3 shows the characterization of Ethyl Violet, an electrostatically bound dye in which:

A) shows the chemical structure of Ethyl Violet; and

B) shows the SERS spectrum of lipid-encapsulated SERS particles using Ethyl Violet as the dye.

FIG. 4 shows an example of the use of a covalently bound dye species 4-fluorobenzenethiol (4-FBT) where the dye is covalently bound to the particle surface through a thiol-gold interaction, and after which the particles are encapsulated with phospholipids, in which:

A) shows the Raman spectrum of the SERS particle bearing the 4-FBT dye (chemical structure inset), and

B) shows the visible absorption spectrum of the same.

FIG. 5 shows the characterization of lipid-encapsulated SERS nanoparticles with 1 mol % rhodamine-lissamine-DSPE (Rho-PE) produced using Method 2, in which:

A) shows the chemical structure of Rho-PE;

B) shows the SERS spectrum of Rho-lipid-coated nanoparticles prepared with Na⁺ showing strong SERS spectrum recognizable as that of rhodamine;

C) shows UV-Vis absorption of Rho-lipid-coated SERS particles prepared with Na⁺. The absorption spectrum of the Rho-lipid-coated particles (black line) is shifted relative to the stock Au (grey solid line), with a shoulder that corresponds to the absorption spectrum of the Rho-PE lipids (grey dashed line); and

D) shows DLS histograms of hydrodynamic radius for stock citrate-coated particles (grey) and Rho-lipid-coated SERS nanoparticles (black outlines). Fits of the DLS data to log-normal centroids report average Rₕ=29.8±0.1 nm and 39.9±0.4 nm for stock particles and Rho-PE SERS particles respectively.

FIG. 6 shows the spectra of Rho-PE/DEC-coated particles produced in the presence of Ca²⁺ in which:

A) shows Raman spectrum of the Ca²⁺-Rho-PE/DEC-coated particles; and

B) shows normalized UV-Vis absorption spectrum of Ca²⁺-Rho-PE/DEC-coated particles (black line), Rho-PE/DEC vesicles (dashed grey line), and stock citrate-coated Au nanoparticles (solid grey line). The first peak in the absorption spectrum of Ca²⁺-Rho-PE/DEC-coated particles at 536 nm was red-shifted by 2 nm relative to stock citrate-coated particles. The second peak at 576 nm coincides with the absorption spectrum of Rho-PE/DEC vesicles, thus the peak was attributed to the absorption of Rho-PE in the vesicle coating.

FIG. 7 shows tryptophan as a Raman active molecule in a lipid-coated SERS nanoparticle in which;

A) shows the chemical structure of Tryptophan (Trp);

B) shows the UV-Vis absorption spectrum of bare gold (grey) and Trp-Lipid-coated SERS particle (black). Peak shifts form 535 nm to 538.6 nm; and

C) shows the SERS spectrum of Trp-lipid-coated SERS nanoparticle which correlates strongly with the SERS spectrum published in Chuang et al.²³ and Aliaga et al.²⁴.

FIG. 8 shows the stability of MGITC-lipid-coated particles and Rho-lipid-coated-particles in which;

A) shows the SERS spectrum of MGITC-lipid-coated particles collected on day of synthesis, 12 days, and 25 days after synthesis; and

B) shows the SERS spectrum of Rho-lipid-coated-particles collected on day of synthesis, and 7 days after synthesis, in which for both cases, particles were stored in water at 4 deg C. between measurements.

FIG. 9 shows an example of the addition of a lipid species with a charged head-group to affect the properties of the lipid-coated particles. Here, the lipid mixture contains DOPC, Sphingomyelin, and cholesterol with the anionic lipid dipalmitoylphosphatidic acid (DPPA) added at 16% molar fraction of total lipids in which:

(A) shows the chemical structure of DPPA, and

(B) shows the UV-Vis absorption spectrum of lipid-coated 60 nm Au nanoparticles that have undergone a chemical reaction with antibodies following encapsulation. The spectrum of particles coated without DPPA (dark grey) exhibits a strong, broad absorption at 680 nm that indicates aggregation has occurred during the reaction, whereas the spectrum of lipid-coated particles containing DPPA (black line) has a greatly reduced absorption at 680 nm indicating that aggregation during reaction has been mitigated. The spectrum of bare 60 nm Au particles (light grey) is shown for comparison.

FIG. 10 Is a schematic of the method of whole antibody conjugation to lipid encapsulated AuNPs. COOH-PEG-DSPE is used as an anchoring molecule to which antibodies are reacted to the surface of the lipid-encapsulated AuNP via EDC/(sulfo)NHS reaction.

FIG. 11 shows the targeting of lipid-coated nanoparticles containing a charged lipid species conjugated to whole antibodies. Lymphocytes extracted from patients with chronic lymphocytic leukemia were treated in vitro with lipid-coated nanoparticles and then washed 5 times by centrifugation. The cells are then fixed with formaldehyde and spun onto glass slides and mounted for microscopy. Slides are visualized by darkfield microscopy and images contain cells and particles. In left-hand column (a) are images of cells treated with particles coated with lipids containing 16% (by mol) DPPG and in column (b) are images for cells treated with particles coated with lipids containing 16% (by mol) DPPA in which:

(A) shows the chemical structures of the DPPG and DPPA;

(B) shows dark field images of lymphocytes treated with Anti-CD19 targeted lipid coated particles that indicate dense labeling of cells by particles;

(C) shows dark field images of lymphocytes treated with Anti-CD19 antibodies to block the CD19 receptors on the surfaces of the cells before being treated with Anti-CD19 conjugated particles. These images indicate particle binding is inhibited by the antibodies, which suggests the particles are CD-19 specific;

(D) shows images of cells treated with unconjugated particles, which show very little binding; and

(E) shows a Raman spectrum of CLL cells labeled treated with lipid-coated, anti-CD19 conjugated particles with malachite green as the dye, which indicates positive labeling of the cells by the SERS particles can be detected by Raman spectroscopy.

FIG. 12 illustrates the example of lipid-coated SERS particles targeted to cells by antibody fragments in which:

(A) is a schematic of how an antibody is fragmented, and conjugated to a lipid-species through maleimide chemistry, and then inserted into a lipid-encapsulated SERS particle;

(B) and C) show dark field images of lymphocytes extracted from a CLL patient exposed to anti-CD20-Fab-targeted lipid coated SERS particles, and unconjugated lipid-coated SERS particles respectively; and

(D) is a Raman spectrum of cell samples treated as in (B) (black solid line) and (C) (grey dashed line).

### DETAILED DESCRIPTION OF THE INVENTION

Generally speaking, the embodiments described herein are directed to methods for functionalizing Surface Enhanced Raman Scattering (SERS) gold nanoparticles by phospholipid encapsulation providing a means of controlling chemical, optical, and targeting properties of the particles. As required, embodiments of the present invention are disclosed herein. However, the disclosed embodiments are merely exemplary, and it should be understood that the invention may be embodied in many various and alternative forms. The figures are not to scale and some features may be exaggerated or minimized to show details of particular elements while related elements may have been eliminated to prevent obscuring novel aspects.

Therefore, specific structural and functional details disclosed herein are not to be interpreted as limiting but merely as a basis for the claims and as a representative basis for teaching one skilled in the art to variously employ the present invention. For purposes of teaching and not limitation, the illustrated embodiments are directed to methods for functionalizing Surface Enhanced Raman Scattering (SERS) gold nanoparticles with Raman reporters adsorbed to surface by phospholipid encapsulation.

As used herein, the terms, “comprises” and “comprising” are to be construed as being inclusive and open ended, and not exclusive. Specifically, when used in this specification including claims, the terms, “comprises” and “comprising” and variations thereof mean the specified features, steps or components are included. These terms are not to be interpreted to exclude the presence of other features, steps or components.

As used herein, the coordinating conjunction “and/or” is meant to be a selection between a logical disjunction and a logical conjunction of the adjacent words, phrases, or clauses. Specifically, the phrase “X and/or Y” is meant to be interpreted as “one or both of X and Y” wherein X and Y are any word, phrase, or clause.

The present invention demonstrates herein embodiments of methods for the encapsulation of metal nanoparticles in lipid vesicles, the stability of lipid-encapsulated nanoparticles, and three avenues by which to include Raman-active species. Encapsulation of the particles by a lipid bilayer is confirmed directly by TEM and indirectly by comparing the particles before and after the encapsulation process with respect to their localized surface plasmon resonance (LSPR, via UV-Vis absorption spectra), hydrodynamic radius (via dynamic light scattering), and colloidal stability in acidic and high ionic-strength conditions. SERS spectra of the lipid-encapsulated particles are reported for five dyes, namely malachite green isothocyanate (MGITC), Ethyl Violet (EV), and 4-Fluorobenzenethiol (4-FBT), rhodamine lissamine DSPE (Rho-PE), and L-tryptophan (Trp), each incorporated by one of the three methods. MGITC and EV represent standard Raman dyes that electrostatically bind to the nanoparticles and have been used frequently in literature to produce SERS nanoparticles. 4-FBT represents thiolated dyes that are covalently bound to the particle surface. Rho-PE is unique because it can be incorporated with the surface functionalization layer (the lipid vesicle) and both can be applied to the particle simultaneously. Furthermore, Rho-PE imparts both fluorescence and Raman activity to the particles. This construct constitutes a potentially useful hybrid Raman/fluorescence probe. The third Raman-active molecule, Trp, has not been widely applied to SERS nanoparticles, though it is interesting as a Raman reporter because it is a natural amino acid, and is inherently biocompatible. Furthermore, it represents a class of dyes that are hydrophobic and incorporate into the lipid layer.

The SERS spectra are monitored over a time period of several weeks to demonstrate stability of the particles. Additionally, the use of ternary lipid mixtures, specifically mixtures of DOPC, egg sphingomyelin, and cholesterol in a 2:2:1 molar ratio (DEC221), which are known to phase-segregate, strengthen the bilayer²⁵ and mimic some of the functional aspects of lipid rafts. This lipid mixture has been studied extensively as a model system for cell membranes. Note that the headgroups of DOPC and egg sphingomyelin are zwitterionic.

The encapsulation of nanoparticles by lipids was achieved by sonicating nanoparticles in a suspension of multilamellar vesicles (MLV) of DEC221 for 45-60 min at 50° C. Sonication of the MLV under these conditions in the absence of particles has been shown to produce unilamellar vesicles (ULV)<100 nm in diameter^{26,27}. This transformation can be observed visually; the MLV suspension appears cloudy, while the ULV suspension appears almost clear, since the size of the vesicles in the ULV suspension has become smaller than the diffraction limit of visible light, and consequently scatters significantly less light than the MLV suspension. Here, it is demonstrated that sonication of the MLV suspension in the presence of metal nanoparticles results in nanoparticles being encapsulated by a lipid bilayer.

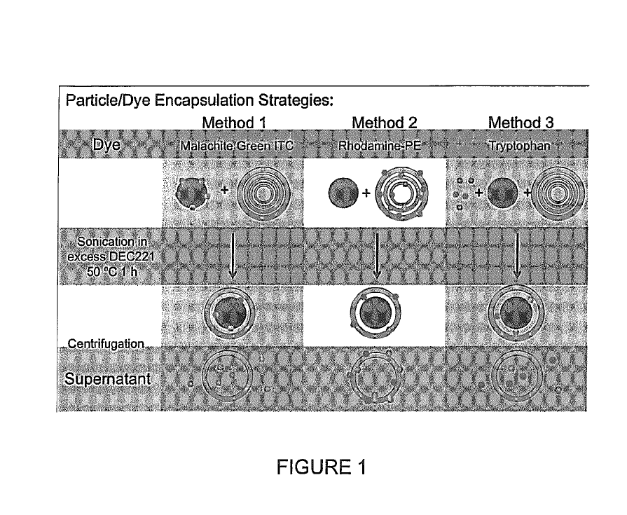

The incorporation of dye molecules was achieved using three methods as illustrated in FIG. 1: (Method 1) dye molecules (MGITC, EV, 4-FBT) were conjugated to the nanoparticle prior to mixing with MLV suspension; (Method 2) a lipid species with a dye covalently-bound to the headgroup of a lipid (Rho-PE) was employed; and (Method 3) an aromatic, hydrophobic amino acid (Trp) was added to the lipid suspension prior to mixing with the particles. The three methods differed by the means by which the dye was added.

The following discloses the details of the three methods of incorporation of dye molecules.

### Au Nanoparticles

Citrate coated Au nanoparticles were purchased from Ted Pella Inc. (Redding, Calif., USA). According to the manufacturer, the particles are nominally 60 nm in diameter. Particles from various batches were employed to ensure that the lipid encapsulation and dye-association processes were not batch-dependent.

### Lipid Preparation

Dioleoylphosphatidylcholine (DOPC), egg-sphingomyelin (ESM), and ovine cholesterol (Chol) (Avanti Polar Lipids, Alabaster, Al, USA) were mixed to a 2:2:1 molar ratio (DEC221) and a final mass of 10.7 mg in a 3:1 chloroform/methanol solution. The solution was divided into 10 aliquots in glass vials and dried under a stream of Argon gas for 1 hr or until the lipids form a film on the bottom of the vials. The vials are then dried under vacuum overnight to remove residual solvent. The dried lipid films are repressurized with Argon gas, capped, and sealed with tape and stored at −20° C. until use.

### Method 1—Malachite Green

An aliquot of DEC221 was thawed and hydrated in MilliQ water to a final concentration of 1 mg/mL and incubated in a 50° C. circulating water bath for 30 minutes with brief vortexing every 10 minutes to form a multilamellar vesicle (MLV) suspension. Meanwhile, aqueous malachite green isothiocyanate was added to Au colloid and stirred for 10 min to facilitate the adsorption of the positively charged ionic dye to the negatively charged, citrate-capped 60 nm Au (forming MGITC-AuNPs), prior to lipid encapsulation. Equal parts DEC221-MLV and MGITC-AuNPs were mixed, while retaining a quantity of MLV suspension in a separate vial for comparison. The nanoparticle/MLV suspension and the retained MLV suspension were sonicated in a bath sonicator for 45-60 min at 50° C., or until the MLV suspension without particles became clear, signifying the formation of small unilamellar vesicles. The vesicles and lipid coated particles were stored at 4° C. until use.

### Method 2—Rhodamine-PE

Rhodamine lissamine-phosphatidylethanolamine (Avanti Polar Lipids) in chloroform was divided into aliquots corresponding to 1 mol % of the DEC221 aliquots. One or more aliquots of the rhodamine-PE was dissolved in chloroform and combined with one aliquot of DEC221, and dried under a stream of Argon for 1 hour, or until the solvent has visibly dried. The vial was then dried under vacuum overnight, repressurized with Argon, capped, and stored at −20° C. until hydration. The Rhodamin-PE was protected from light by reducing the room lighting as much as practical, wrapping the vials in aluminum foil, and keeping the vials covered whenever practical.

Rhodamine-PE/DEC MLVs were made as described in Method 1. The final rhodamine-PE concentration was 5 mol % total lipid. In some cases, NaCl (10 mM final concentration) or CaCl₂ (2 mM final concentration) was added to the MLV suspension. The MLV was divided into two aliquots and placed in the sonicator at 50° C. An equal volume of the AuNP suspension was added to one vial, and sonication was continued until the lipid vesicle suspension was clear. Both the vesicles and the lipid coated particles are then sealed and stored at 4° C. until use.

### Method 3—Tryptophan

The DEC221 MLV solution was prepared as described in Method 1, except the lipid aliquot was hydrated in a 1 mM L-tryptophan solution. Equal parts Trp-MLV and AuNP were combined in a vial, and a portion of the MLV was retained in a separate vial. As before, both were sonicated at 50° C. for 45-60 min or until the pure lipid suspension became clear. Vesicles and lipid-coated particles were sealed and stored at 4° C. until use.

All dye-lipid-particle products underwent two cleaning steps before any measurements were made. Each cleaning step involved centrifugation of the particle/vesicle suspension at 4500 RPM for 5 minutes using a desktop microcentrifuge. This settled the particles to the bottom of the microcentrifuge tubes, and the supernatant was removed and retained in a separate container. The particles were then resuspended to the same concentration in 18 MO-cm water.

### Characterization

### Raman Spectroscopy

An inverted microscope (Nikon TE2000) was used to focus the CW 632.8 nm HeNe (15 mW) laser beam onto the sample, in episcopic configuration. The laser beam was collimated before entrance into the optics of the objective (S Plan Fluor ELWD 40×, NA 0.6). The Rayleigh scattering from the sample at the wavelength of the laser line is blocked from entering the monochromator by a notch filter (Λ>645 nm). An achromatic doublet lens (f/6.6) focuses the Raman scattered light onto the monochromator slit. The Acton SP2560 Czerny-Turner monochromator (f 6.5) had a triple grating turret (1200 g/mm, 750 blaze wavelength used to collect displayed spectra). The monochromator was connected to a Princeton Instruments PIXIS BR 400 CCD detector with a 1340×400 pixel array that was Peletier cooled to 75 degrees Celsius below room temperature.

### UV-Vis Spectroscopy

Stock particles and washed lipid-coated particles were placed in a 1 cm-path-length cuvette. A Cary 5000 UV-Vis spectrometer (Varian Inc. Palo Alto, Calif., USA) was used to collect the absorption spectra of the particles using 18 MΩ-cm water as a reference. Spectra were normalized by shifting the baseline to zero, and scaling the data linearly so that the maximum value was 1.

### Dynamic Light Scattering

DLS was performed using a DynaPro/Protein solutions DLS machine (Wyatt Technologies Corporation, Santa Barbara, Calif., USA). Particle dilution and laser power were adjusted for optimal signal. Typically, particle concentrations were diluted 1:20 compared to stock concentrations. The thermal stator was set to 25° C., and samples were allowed 2 minutes to equilibrate with the thermal stator before measurements were collected.

In all cases, the particles were washed of excess lipids by two centrifugation steps. Since unbound dye molecules are discarded with the supernatant, the SERS spectra collected from the washed particles confirms the incorporation of the dye species. In the case of Rho-PE particles, the SERS spectrum of the particle confirms the presence of both the dye and the lipid coating on the particle since the dye is anchored to the bilayer, and unbound lipids and dye are removed by centrifugation.

### Transmission Electron Microscopy

Samples for transmission electron microscopy (TEM) were prepared by placing a drop of nanoparticle solution on a carbon-coated copper grid and wicking away excess liquid. Grids were then air-dried. TEM images were obtained using a Hitachi H-7000 TEM instrument operated at 75 kV. Image analysis was performed using ImageJ software. The length-scale was calibrated against the scale bar, and the thickness of the bilayer was measured in software. The bilayer was measured in 5 places around each particle, and 5 particles were measured in total to determine the average thickness.

### Characterization of Lipid-Encapsulated SERS Particles

### Method 1:

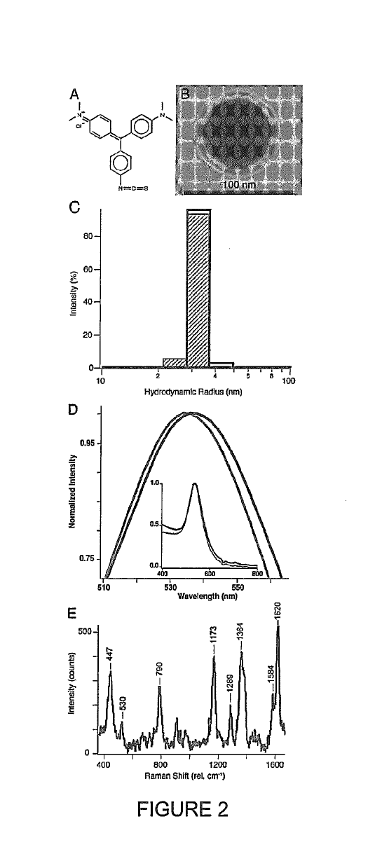

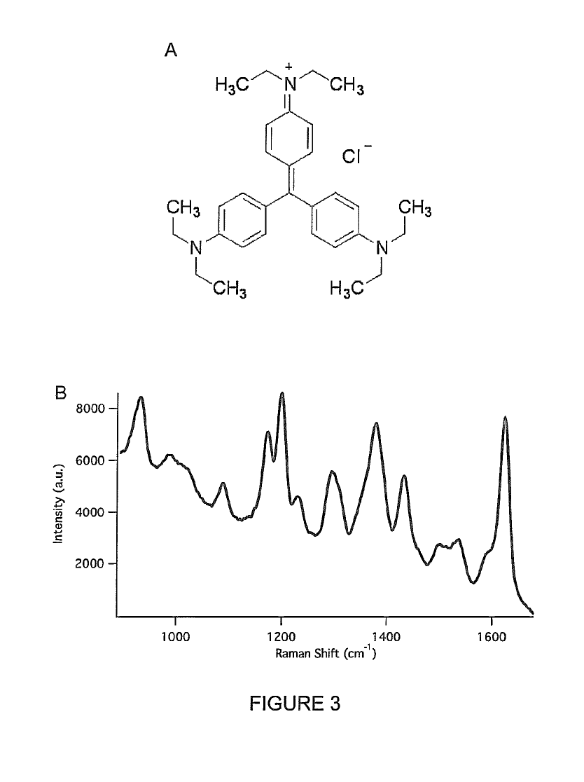

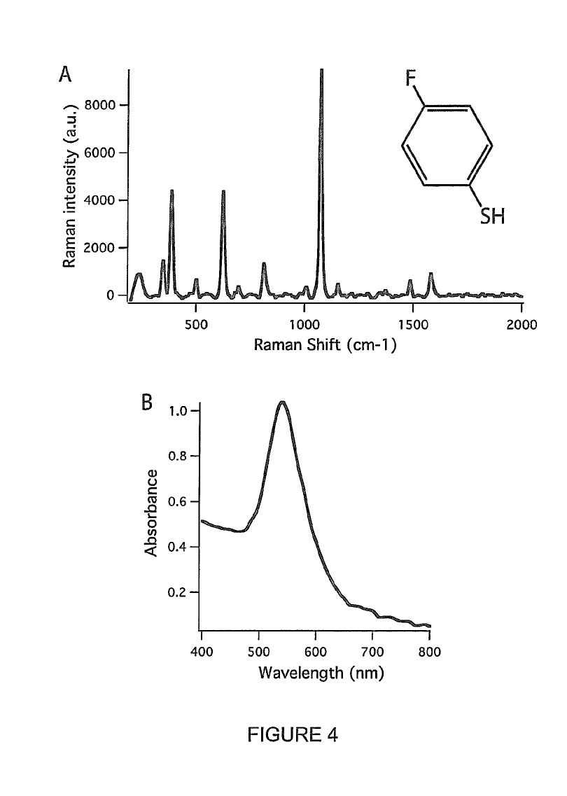

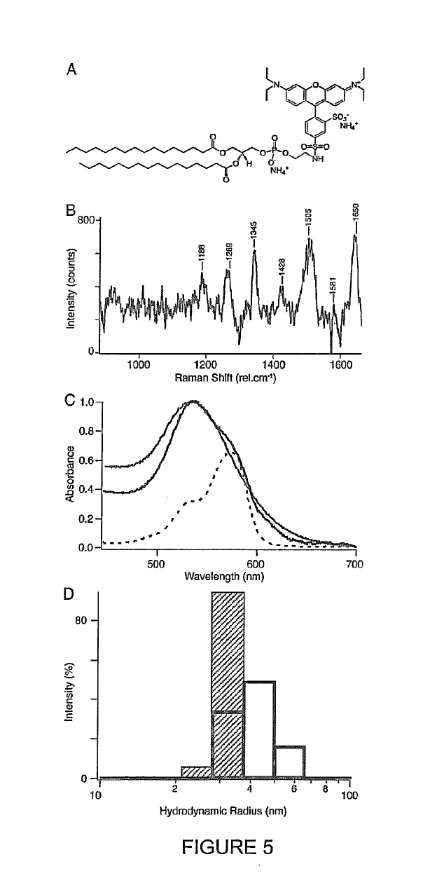

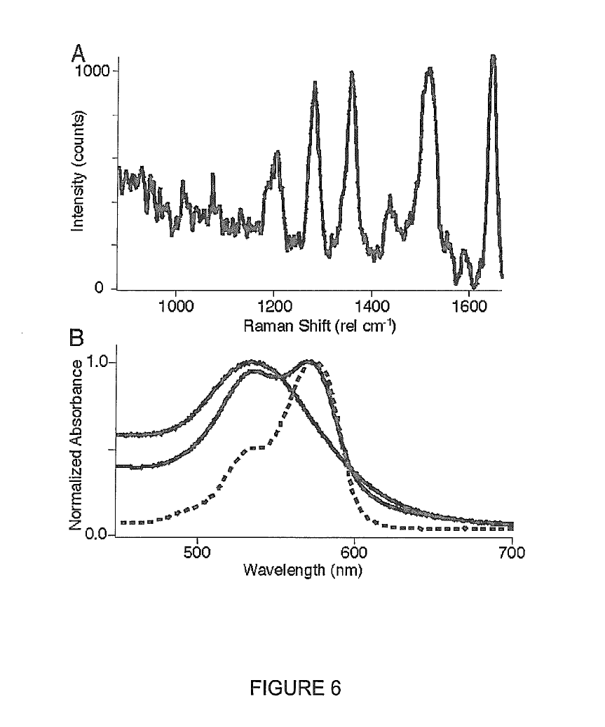

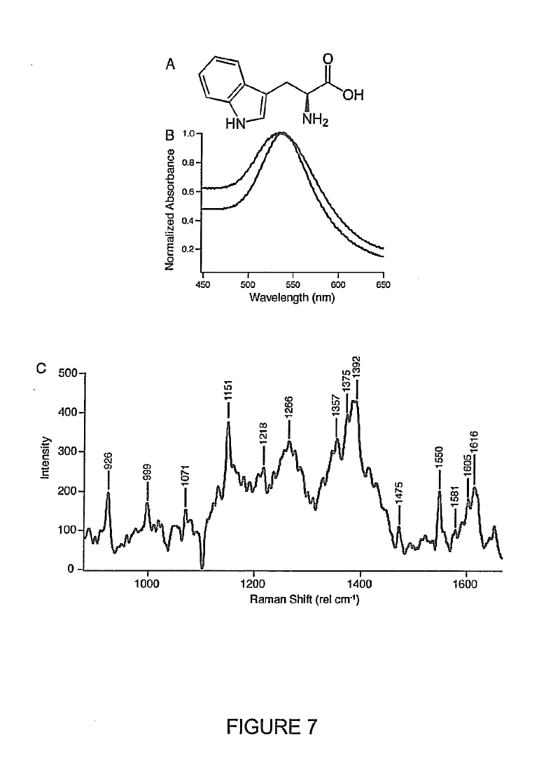

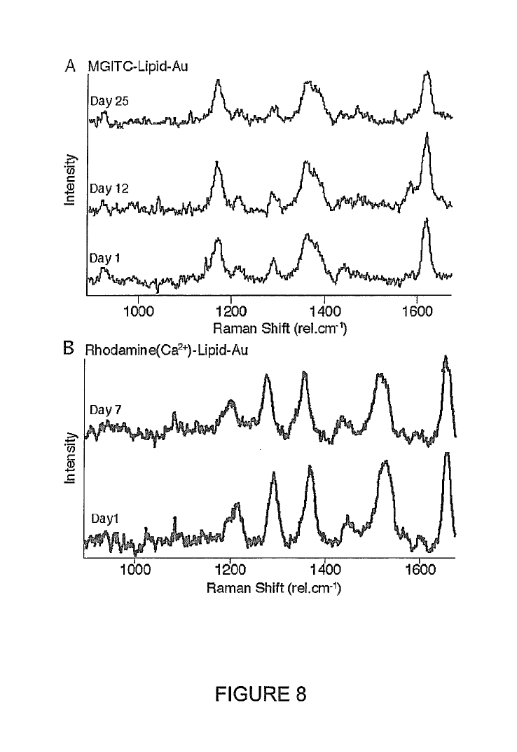

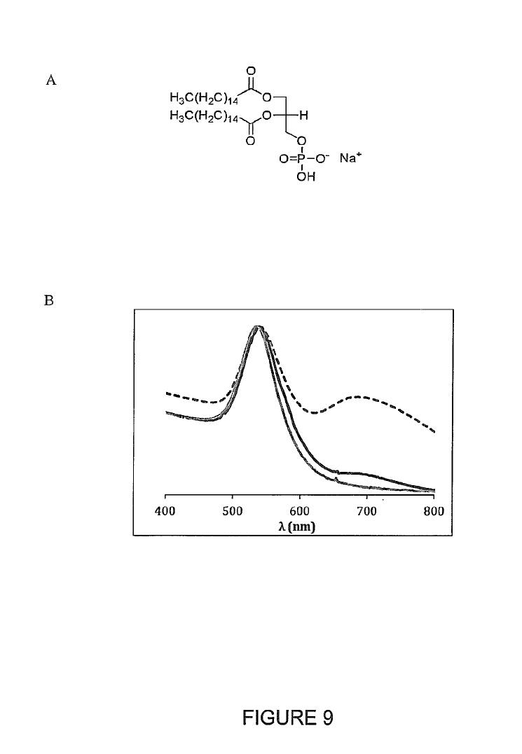

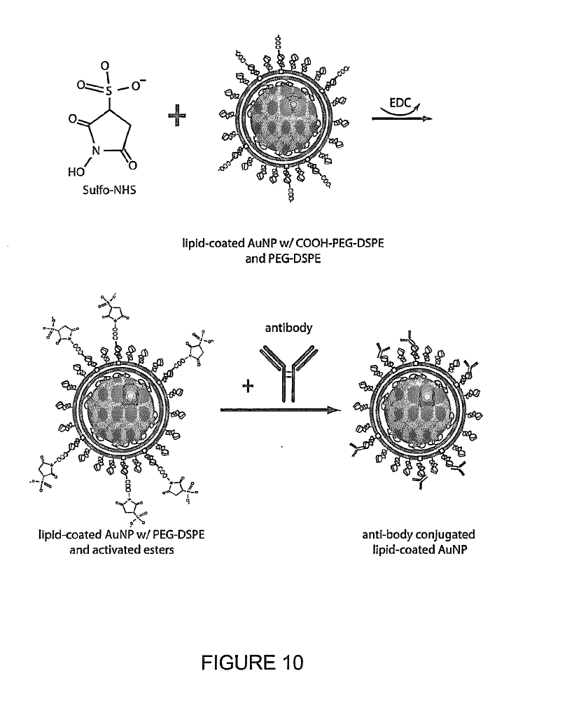

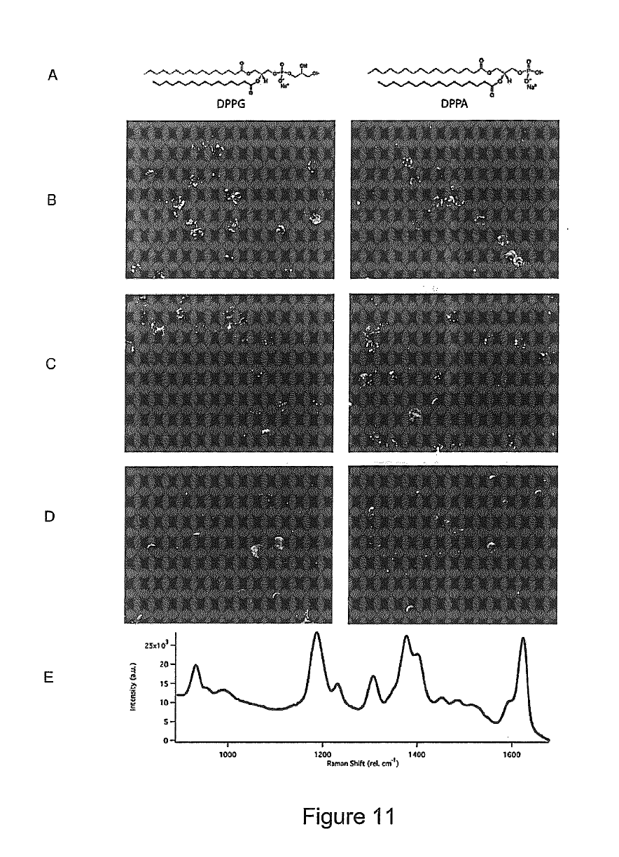

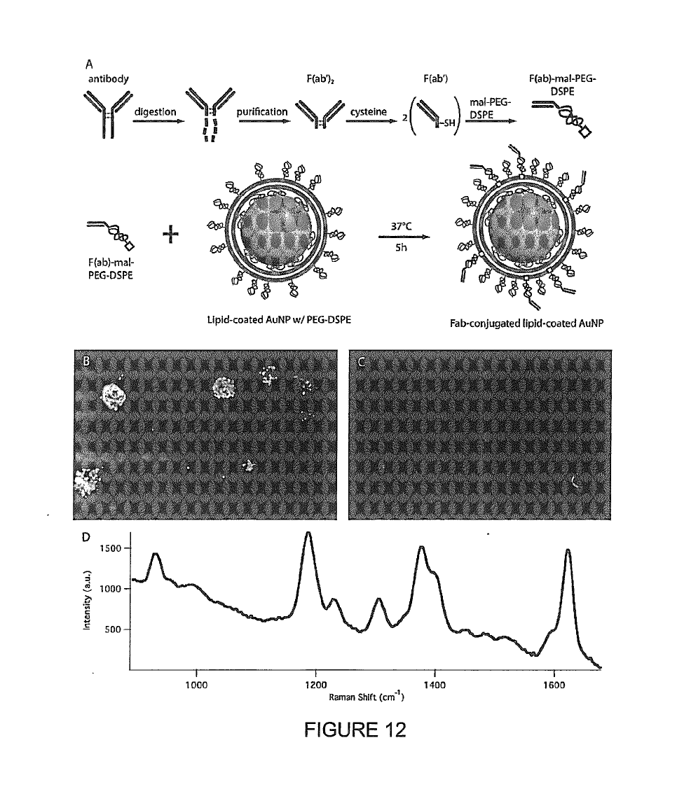

MGITC (structure: FIG. 1A), was associated to the particle prior to lipid encapsulation. The lipid encapsulation serves to protect the dye/particle conjugate from flocculation or dissociation. The lipid layer was observed directly by TEM, as shown in FIG. 2B, where it appeared as a diffuse ring around the dark nanoparticle, with an average thickness of 4.8 nm, which is consistent with the expected size increase for the formation of a bilayer²¹. The hydrodynamic radius of the lipid coated particles, compared to the stock citrate-coated particles measured by DLS (FIG. 2C) were consistent; the centroids of log-normal fits to the histograms suggest that the average hydrodynamic radius of the particles has increased from 29.8±0.1 nm to 34.1±0.5 nm due to the presence of the lipid bilayer.

The presence of the lipid bilayer was further confirmed by the UV-Vis absorption spectrum of the particle whose main localized surface plasmon resonance (LSPR) absorption red-shifted from 534 nm to 538 nm when compared to the stock particles due to the addition of the dielectric lipid layer (FIG. 2D). This shift was significant compared to the 2 nm shift widely reported for the addition of bulky polyethylene glycol to the particle surface'. FIG. 2E is the SERS spectrum of the lipid-encapsulated particles, which can be identified as the SERS spectrum of MGITC reported elsewhere^{7,29,28}, indicating that the MGITC remains associated with the metal particle following the sonication with lipids.

Table 1 below shows assignment of observed SERS bands of MGITC based on Lueck et al.²⁸ (Lueck, H. B.; Daniel, D. C.; McHale, J. L. J. Raman Spectrosc. 24, 363-370). “Band” column is the observed wave-number of a peak. (*) indicates that correlation of band position with ref ²⁸ is >5 cm⁻¹, and that the assignment is the closest match. “Chemical group” and “mode” columns indicate the chemical group and type of vibrational mode assigned by Lueck et al.²⁸ Multiple assignments to a band are separated by semicolons. Parentheses indicate location of the chemical group within the molecule.

TABLE 1
Assignment of observed SERS bands of MGITC based on Lueck et al.²⁸

| Band (cm⁻¹) | Chemical Group | Mode |
|---|---|---|
| 447* | Benzene | ring deformation (out of plane) |
| 530 | Benzene | ring deformation (in plane) |
| 791* | C—H (Benzene) | Wagging |
| 1173 | C—H | Rocking |
| 1289* | C—C; C—C—H | rocking; rocking |
| 1364 | —N | Stretch |
| 1584* | ring | Stretch |
| 1620 | —N; C—C | stretch; stretch |

To further illustrate examples of this method, the cationic dye Ethyl Violet (EV), which unlike MGITC, does not contain an isothiocyanate group, was associated with the particles prior to encapsulation. FIG. 3A illustrates the structure of Ethyl Violet and FIG. 3B shows the SERS spectrum of lipid-encapsulated EV-Au-NPs.

An additional example illustrates that not only can dyes be bound electrostatically to the particle as in MGITC and EV, but they can also be covalently bound to the AuNP surface prior to encapsulation. 4-fluorobenzenethiol (4-FBT) represents a class of molecules that contain a Raman-active aromatic group as well as a thiol group, which can covalently bind to metallic and semiconductor surfaces. FIG. 4 illustrates the use of 4-FBT as a dye in lipid-encapsulated SERS particles. FIG. 4A is the SERS spectrum of the particles (dye structure inset) and FIG. 4B is the UV-Vis absorption spectrum that indicates no aggregation.

### Method 2:

A small amount (˜5 mol % total lipid) of an appropriate dye-labeled phospholipid (rhodamine-PE), was incorporated into the DEC221 aliquots thus, surface encapsulation and dye conjugation of the particle was achieved simultaneously. By extension, lipid species with targeting moieties may in principle be incorporated simultaneously in much the same way. Here, a phospholipid with a rhodamine-lissamine modified headgroup (structure: FIG. 5A) was chosen because it is commercially available and because rhodamine dyes have been shown to have strong SERS cross-sections³⁰⁻³². The use of dye-labeled lipids was advantageous because the dye was covalently bound to the lipid, and thus the acquisition of a SERS spectrum of the dye following sonication and centrifugation of the particles served as an additional means of confirming the presence of the bilayer next to the particles. This is because dissolved lipids and even vesicles can only be settled from suspension under very high centrifugation speeds compared to the dense gold nanoparticles³³. Separation of the particles from the supernatant separated unbound lipids from the encapsulated particles. Presumably, only those lipids bound to the particle were close enough to the particle's surface to contribute significantly to a detectable SERS signal.

Note that the rhodamine-labeled lipids have a net-negative headgroup charge. Because the as-purchased, citrate-coated particles also have a net-negative charge, Rho-PE lipids in the encapsulating bilayer are primarily segregated to the outer leaflet, as has been demonstrated for other charged lipids on charged surfaces³⁴⁻³⁷. In this orientation, SERS excitation is expected to be less efficient. Initial attempts to incorporate Rho-PE lipids produced particles with fluorescence spectra but inconsistent SERS spectra from batch to batch. It was therefore hypothesized that the addition of positive counter ions in the form of monovalent sodium may act to neutralize these charges and allow the rhodamine-conjugated lipid to reside in the inner leaflet. FIGS. 5B, 5C and 5D show the SERS spectrum, the UV-Vis spectrum, and DLS data for Rho-PE/DEC-coated particles prepared in the presence of 10 mM NaCl.

The SERS spectrum, which has had the fluorescence background subtracted, is recognizable as that of rhodamine^{29,40,39,38}. This indicates that the Rho-PE lipids have been incorporated into the bilayer coating, and that they reside close enough to the particle surface to contribute to the SERS spectrum. Note that the particles were separated from unbound lipids and vesicles by centrifugation.

Table 2 below shows the assignment of observed SERS bands of Rho-PE based on Zhang et al.³⁹ (Zhang, J.; Li, X.; Sun, X.; Li, Y. J. Phys. Chem. B 2005, 109, 12544-12548) (Ag SERS/rhodamine B) and based on Jensen et al.³⁸ (Jensen, L.; Schatz, G. C. J. Phys. Chem. A 2006, 110, 5973-5977.) (Normal/Resonant Raman/Calculations on rhodamine 6G). “Band” column is the observed wave-number of a peak. (*) indicates that correlation of band position with Jensen et al. is >5 cm⁻¹, and that the assignment is the closest match. (†) indicates correlation with Zhang et al.³⁹ is >5 cm⁻¹ and that the assignment is the closest match. “Chemical group” and “mode” columns indicate the chemical group and type of vibrational mode assigned by Zhang et al.³⁹ and Lueck et al.²⁸. Multiple assignments to a bands are separated by semicolons. Parentheses indicate location of the chemical group within the molecule.

TABLE 2
Assignment of observed SERS bands of Rho-PE based on Zhang et al.³⁹

| Band (cm⁻¹) | Chemical Group | Mode |
|---|---|---|
| 1188^{†} | Xanthene; C—H | deformation; bending |
| 1261-1269*^{†} | Xanthene | ring breathing |
| 1345*^{†} | Xanthene | ring stretching |
| 1428 | not assigned | not assigned |
| 1505/1513* | Xanthene; C—N | ring stretching; stretch |
| 1581^{†} | Xanthene | ring stretching |
| 1650 | Xanthene; C—H | ring stretching; rocking |

The UV-Vis absorption spectrum of the Rho-Lipid coated particles (black line in FIG. 5C) appeared red-shifted by 3 nm relative to that of the citrate-coated gold (solid grey line). This shift was similar in magnitude to that observed for Method 1 products. A shoulder was also observed on the red-side of the absorption band, which corresponds well with the absorption of the Rho-PE-containing lipid vesicles (dashed grey line). Log-normal fits to the DLS histograms reported an average hydrodynamic radius of 39.9±0.4 nm, compared to 29.8±0.1 nm for the stock citrate-coated particles. The DLS data suggested a larger bilayer than the particles produced by Method 1. This may have been due to the net-surface-charge of the particle imparted by the net-negative Rho-PE lipids in the bilayer, which contribute to a large electrical double layer, or a structured hydration layer.

An alternate strategy employed to counteract the charge repulsion between the Rho-PE and the citrate coating on the Au nanoparticle was to prepare the particles in the presence of Ca²⁺. The divalent nature of the calcium cation acts to bridge the negative surface charges with the negative headgroup charges. Calcium has been widely employed to promote the fusion of planar lipid bilayers on negatively charged surfaces such as mica. Rho-PE/DEC-coated particles produced in 2 mM CaCl₂ exhibited strong Raman signals, as shown in FIG. 6A. As with the Rho-PE/DEC-coated particles produced in the presence of Na⁺, the particles are separated from excess lipids by two centrifugation steps and the spectrum of the rhodamine-containing vesicles alone was subtracted from the particle spectrum to remove the fluorescence background. The observed peaks are consistent with the SERS spectra of rhodamine incorporated in the presence of Na⁺.

The UV-Vis absorption spectrum of the Ca²⁺-Rho-lipid-coated particles (black line, FIG. 6B) exhibited a more pronounced contribution from the rhodamine dye than was the case for the particles prepared in the presence of Na⁺. What appeared as a shoulder in the case of particles prepared in the presence of Na⁺, appears as a second peak at 576 nm in the case of particles prepared in the presence of Ca²⁺. This second peak coincides with the main absorption peak of the Rho-PE-containing vesicles (dashed grey line). The other peak at 536 nm is consistent with the absorption peak of lipid-coated particles measured in the Na⁺ case. DLS data from the Rho-PE-DEC-particles encapsulated in the presence of Ca²⁺ were inconclusive (not shown), since the regularization algorithm could not provide reliable hydrodynamic radii.

### Method 3:

Soluble dyes were also co-encapsulated with the particles during sonication. In this case, the amino acid L-tryptophan (structure: FIG. 7A) was dissolved along with the lipids prior to forming the MLV solution. Upon sonication with the particles, Trp likely co-encapsulated with the particles. Nonetheless, due to the partition coefficient of Trp⁴¹, the possibility of Trp partitioning into the lipid layer cannot be ruled out. While Trp has been found to localize at the lipid-water interface when incorporated into membrane proteins⁴², the exact configuration of the Trp in this construct has not been studied. Nevertheless, the nanoparticles prepared in this fashion exhibited a 3.6 nm shift in the UV-Vis/LSPR spectrum, which was comparable to particles made by the other two methods. The Raman spectrum of the Trp particles is shown in FIG. 7C. The spectrum of the lipid-coated particle is consistent with the Raman spectrum of Trp reported in literature^{23,24}. A solution-phase Raman spectrum of a Tryptophan solution, at the same concentration at which it was added to the lipid suspension was not detectable using the same laser power and integration time. This suggests that although the tryptophan SERS signal was weaker than that of the MGITC-lipid-particle sample, the observed spectrum nevertheless indicates a significant enhancement of the Raman signal. FIG. 7B) shows the UV-Vis absorption spectrum of bare gold (grey) and Trp-Lipid-coated SERS particle (black). Peak shifts form 535 nm to 538.6 nm.

Based on the band assignments summarized in reference²³, the bands observed here can be assigned to various vibrational modes of the molecule. Table 3 below shows assignment of observed SERS bands based on Chuang et al.²³ (Chuang, C.; Chen, Y. J. Raman Spectrosc. 2009, 40, 150-156.). “Band” column is the observed wave-number of a peak. (*) indicates that correlation of band position with Chuang et al. is >5 cm⁻¹, and that the assignment is the closest match. “Chemical group” and “mode” columns indicate the chemical group and type of vibrational mode assigned by Chuang et al.²³. Multiple assignments to a band are separated by semicolons. Parentheses indicate location of the chemical group within the molecule.

TABLE 3
Assignment of observed SERS bands based on Chuang et al.²³

| Band (cm⁻¹) | Chemical Group | Mode |
|---|---|---|
| 926 | C—COOH⁻; NH3⁺; CH₂ | stretching; rocking; rocking |
| 999 | Indole | ring breathing |
| 1071 | NH3⁺; (C—)H | rocking; bending |
| 1151 | H (benzene) | Scissoring |
| 1218 | pyrole, C—COOH⁻ | stretching; stretching |
| 1266* | H(-indole); CH; H(-methyl) | rocking; bending; bending |
| 1357* | CH; H(-methyl) | bending, bending |
| 1375* | CH₂; CH | wagging, bending |
| 1392 | COO⁻ | stretching (symmetric) |
| 1475* | CH₂ | Scissoring |
| 1550 | indole | Stretching |
| 1581 | NH3⁺ | Scissoring |
| 1605 | indole | Stretching |
| 1616* | COO⁻ | stretching (antisymmetric) |

The stability of the particles can be assessed by observing the SERS spectra over time, since precipitation of the particles or dissociation of the dye from the particles would result in a reduction in the Raman intensity. The Raman spectra of the MGITC-lipid-coated-particles were recorded after 12 and 25 days of storage at 4° C., and show no significant signs of signal change, indicating the stability of the particles over time. These spectra, offset for clarity, are shown in FIG. 8A. The spectra for rhodamine-lipid-coated-particles prepared in the presence of Ca²⁺ exhibited stable SERS spectra over the course of 7 days. The spectra of Rho-lipid-particles at 1 and 7 days are shown in FIG. 8B, with the spectra offset for clarity.

The colloidal stability was also tested by subjecting both the stock citrate-coated nanoparticles and the lipid-encapsulated particles to several ionic and pH conditions, using the same particle concentrations. An additional sample, containing just the ULV suspension was also subjected to the same conditions as a control. For these tests, a fluorescent Nitrobenzoxadiazol (NBD)-tail-group-labeled phosphocholine lipid was incorporated into the lipid mixture at 0.2 mol % because this species imparts a bright green fluorescence that helps visualize the ULV suspension. It was also included in the particle-lipid suspension at the same concentration for consistency. Because the dye is conjugated to the tail-group, the chemical properties of the head-goups remain the same as the other components of DEC221.

Table 4 summarizes of test conditions and observations. For each test, small aliquots of each suspension were tested independently, with the final concentration of the lipids in the ULV suspension, and the nanoparticles in the “stock” solution being nominally the same as those in the lipid-encapsulated particle suspension.

TABLE 4
Summary of colloidal stability test: Lipid-coated particles, lipid vesicles,
and stock particles are subjected to high salt and low pH conditions.
Descriptions of the suspensions are summarized.

| | Lipid- | Control 1: | |
|---|---|---|---|
| | Encapsulated | Particle | |
| | Particles | Only | Control 2: Lipid only |
| Treatment | Clear, pink | Clear, pink | Greenish-yellow (NBD), |
| a) Water | solution | solution | faintly cloudy |
| Treatment | Clear, pink | Clear, | Less colourful |
| b) Acetic Acid | solution | Colourless | |
| Treatment | Paler pink | Clear, | Greenish-yellow, faintly |
| c) CaCl₂ | | Colourless | cloudy |
| Treatment | Clear, pink | Clear, pink | Greenish-yellow, faintly |
| d) NaCl | solution | solution | cloudy |
| Treatment | Clear, pink | Clear, pink | Greenish-yellow, faintly |
| e) PBS | solution | solution | cloudy |

The suspensions were subjected to 5% acetic acid, 10 mM CaCl₂, 10 mM NaCl, and PBS (50 mM monvalent salts), to test the resistance to acidic pH, divalent cations, and various monovalent salt concentrations. Baseline descriptions of the stock particles, lipid encapsulated particles, and ULV suspension were taken in ultra-pure water. See Table 4, treatment (a).

In the first test (condition b), the pH of the environment for each of the three suspensions was lowered by adding acetic acid to a final concentration of 5% v/v. At neutral pH, the stock particles are protected from aggregation by mutual electrostatic repulsion provided by the anionic citrate coating. The addition of acid to the environment is expected to cause the protonation of citrate, and subsequently the flocculation of the nanoparticles. Indeed this was observed; the colour of the suspension of citrate-coated particles changed from pink to colourless, indicating that the nanoparticles had flocculated. Conversely, the lipid-coated particles were not visibly affected by the acid, suggesting that the particles were protected from flocculation by means other than protonatable electrostatic repulsion, such as steric repulsion of the lipids, or mild induced dipole interactions between zwitterionic headgroups.

In principle, increasing the ionic strength of the environment of the particles can shield the electrostatic interactions that stabilize particle suspensions. Specifically, divalent cations are known to bridge two anionic charges. Ca²⁺ ions, even in modest concentration, are expected to cause aggregation of the citrate-coated particles by this bridging mechanism. Similarly, Ca²⁺ is also known to electrostatically bridge lipid headgroups, which has been exploited to induce phase segregation⁴³ and also fusion of vesicles on anionic surfaces⁴⁴. Ca²⁺ was therefore expected to affect the stability of lipid-coated particles as well. The findings for condition-c were consistent with these expectations. Ca²⁺ caused the citrate-coated particles to aggregate as evidenced by the loss of colour of the suspension. In the case of the lipid-coated particles, the effect was less dramatic. The pink colour became less intense, but unlike the citrate-coated particles, the colour of the lipid-coated particles was not lost completely. The reduced aggregation effect might have been due to weaker interaction between the lipid headgroups and calcium, than between the negatively charged citrate and positively charged calcium, which is expected because the charges on the zwitterionic lipids are only induced transiently. Predictably, the observed cloudiness of the ULV suspension increased.

Similar concentrations of Na⁺ ions had little effect on the stock nanoparticles. Moderate concentrations of Na⁺ would not be expected to affect either the ULVs or the lipid-coated particles. Likewise, the addition of PBS, a higher concentration of salt with additional large molecular counter ions (phosphate) did not have an observable affect on any of the test or control suspensions.

The lipid mixture used to encapsulate the particle may be phospholipid alone, sphingolipid alone, or phospholipid and sphingolipid, or phospholipid and sterol, or sphingolipid and sterol, or phospholipid and sphingolipid and sterol, where phospholipids can be one type or a mixture of phospholipids, and likewise for sphingolipids and sterols. An embodiment used herein was a mixture that included one phospholipid, predominantly one sphingolipid, and cholesterol that is known to form lipid rafts. It is known from the literature that lipid rafts are known to affect membrane signaling.

The phospholipids may have one or more hydrocarbon chain(s) being any one or combination of saturated, monounsaturated, and polyunsaturated. Similarly the sphingolipids may have one or more hydrocarbon chain(s) being any one or combination of saturated, monounsaturated, and polyunsaturated.

Among phospholipid and sphingolipids, the headgroups could be phosphatidyl choline (PC), phosphatidyl ethanolamine (PA), phosphatidyl inositol (PI), or phosphatidyl glycerol (PG). The lipid headgroups can be positively charged, negatively charged, zwitterionic, or neutral. The lipids may have chemically modified tail groups and/or chemically modified headgroups (for example, biotin labled lipids, dye-labeled lipids, lipids with reactive species, etc).

The phospholipids may be sourced naturally (ex: lung surfactant, or egg), or be completely synthetic. Detergents can be included.

The use of acids and/or salts may be employed to control the distribution of charged lipids in the bilayer. The present method may use gold particles that are capped by positively (ex: CTAB) or negatively (ex: citrate) charged ligands with any and all combinations of lipids.

In summary, the lipid/dye encapsulation of gold nanoparticles constitutes a flexible platform by which to control the surface properties and SERS spectra of metal nanoparticles for diagnostic and/or therapeutic applications. We have demonstrated the encapsulation of commercially available, citrate-coated gold nanoparticles by model lipid bilayers, along with three methods of incorporating Raman-active molecules. Malachite green, rhodamine-lissamine DSPE, and tryptophan were employed to demonstrate the three methods. SERS spectra of the three dye-lipid-particle constructs were observed, and lipid bilayer encapsulation was confirmed by TEM imaging, dynamic light scattering, and the endogenous UV-Vis/LSPR spectroscopy of the particles. MGITC is a widely used standard for producing SERS nanoparticles. Rho-PE is unique because it was incorporated with the lipids and thus dye conjugation and lipid encapsulation of the nanoparticle was performed in one step. In addition to SERS, the resulting particles exhibited fluorescence emissions as well. Trp is a unique Raman probe because it is a natural amino acid, and is inherently biocompatible. The use of a ternary lipid mixture offers the possibility of enhanced mechanical stability; control of the membrane modulus also controls the driving force for NP-lipid mixing. Furthermore, rafts offer a route to concentrate antibodies and an opportunity for creating anisotropic binding. A large body of knowledge exists in literature for the protection and targeting of vesicles and liposomes in the context of drug delivery. Combining existing targeting strategies with the encapsulation and dye-association techniques demonstrated here constitutes a new system for the development of SERS nanoparticles for medical diagnostics and therapeutics.

The present invention provides several unique and advantageous features. Using phospholipids, SERS gold nanoparticles have been encapsulated to impart stability and prevent aggregation. Lipid encapsulation improves the biocompatibility and blood circulation time of nanoparticles.

The encapsulated SERS nanoparticles can be conjugated to targeting antibodies, peptides or other ligands to target the cell surface antigen, which are clinically relevant surface biomarkers of disease.

Added cholesterol can enhance the toughness of the particle. It can also be used to impart special stability to introduced integral membrane proteins, transmembrane peptides, and similar species. Major advantages of lipid encapsulation using the methods disclosed herein include: particle aggregation is prevented; there is no observable desorbing of the dyes bound to the nanoparticles; the methods provide a flexible and adaptable platform in which to place targeting ligands either covalently attached to modified phospholipid, attached to a transmembrane peptide or protein, or covalently grafted to the underlying gold particle. Certain ligands may be used to control endocytosis, or to promote fusion of the vesicle with a cell, and allow subcellular markers to be targeted.

The present invention provides a flexible and adaptable platform into which to place dye molecules for SERS—either covalently attached to modified phospholipid, attached to a transmembrane peptide or protein, or covalently grafted to the underlying gold particle. Lipid encapsulation of gold nanoparticles reduces recognition from immune system, avoiding the problem of rapid clearance by reticuloendothelial system.

Lipid encapsulation is adaptable to a variety of lipid species with various acyl chain saturation states (ex: saturated, mono-unsaturated, poly-unsaturated, functionalized, etc), and/or a variety of head group species, (ex: charged, uncharged, zwitterionic, functionalized, etc), as well as a variety of lipid species such as sphingolipids, sterols, lipopolysaccharides, etc, either naturally occurring or synthetic, and/or mixtures and/or combinations therein. The flexibility to incorporate various lipids and/or mixtures is advantageous in controlling and optimizing the properties of the encapsulation and/or adapting them to various applications.

For example, some lipid mixtures such as DOPC/Sphingomyelin/Cholesterol are known to phase segregate into raft-like domains which are known to mimic the behaviour of natural cell membranes in some situations, and may be advantageous or necessary for hosting some membrane proteins or ligands, and facilitate the targeting or adhesion to cells in some situations. The same lipids mixed in different ratios are known to affect the physical and mechanical properties of the lipid layer. Thus, the composition of the lipid layer may be changed to tailor the properties of the layer to various applications.

For another example, lipids with charged headgroups such as phosphatiyl glycerol (PG) and phosphatidic acid (PA) can be used to provide additional stability through electrostatic repulsion. FIG. 9 shows an example of how adding DPPA to the lipid mixture can help prevent aggregation of particles during a chemical reaction of the particle. Furthermore, the use of lipids with polyethylene glycol modified head-groups can be used to provide additional steric stability and improve biocompatibility.

The types and quantities of sterols is known to affect the physical and mechanical properties of a lipid layer. Thus, sterol quantities may be adjusted to control the properties of the layer for various applications. Changing lipid types allows one to achieve different curvatures around different sized NPs. For example, by changing the relative headgroup and lipid-group size. This may be advantageous for encapsulating particles of various shapes. The lipid layer may be used encapsulate more than one particle. This has potential to augment the SERS signal due to the enhanced field known to exist between closely associated particles. The lipid layer may act to stabilize particle aggregates. Lipid encapsulation can be achieved by a variety of methods and/or under a variety of conditions to suit applications. Examples below illustrate the use of a bath sonicator, though other methods such as tip-sonication, extrusion through porous membranes, and the Mozafari method may be used. In the context of this invention, the step of “agitating” a solution containing nanoparticles, and encapsulating agent such as phospholipids and a organic dye such as a SERS reporter means to agitate the solution so as to cause the agent to form a layer around the nanoparticles and dye, and includes sonication (including tip-sonication), extrusion through porous membranes, and the Mozafari. Sonication is the preferred method.

Lipids may be dispersed in various media with various salts, buffers, or other additives to suit applications. An example below illustrates the addition of tryptophan to the dispersion media, which acts as a Raman reporter for SERS. Other additives, materials, or solvents may be used to adapt to specific applications. Changing the conditions of the encapsulation step can produce lipid capsules of different sizes. Specifically, lipid capsule size can be controlled by changing the lipid composition, or by changing the lipid encapsulation conditions and/or method. Size control may be advantageous to take advantage of the enhanced permeability and retention (EPR) effect, or to control the inclusion of water (or other dispersion material) into the capsule.

The lipid encapsulation may be used to deliver particles with various functionalizations to the interior of the cell. As an example, lipid-bound ligands can target a lipid capsule to a particular cell of interest, where the capsule contains a variety of particles functionalized to bind to various sub-cellular targets, and delivers its payload to the interior of the cell via endocytosis or fusion with the cellular membrane.

Other industrial applications of the present invention including applying the technique as assays for both nucleic acids and proteins; in vitro diagnostic, in vivo imaging of other medical pathologies (cardiac, renal, neuronal), in vitro and in vivo detection of blood borne pathogens; agricultural testing; food safety testing; animal health testing; and multiplexed assays for immunodiagnostics, molecular diagnostics, and proteomics, for patient and hospital lab testing, to population wide screens, to mention just a few examples.

Several experiments have been conducted to illustrate these applications. Specifically, the conjugation of these particles to antibodies and antibody fragments, and the use of these conjugated particles to label lymphocytes in blood fractions extracted from Chronic Lymphocytic Leukemia (CLL) patients.

FIG. 10 is an illustration of the chemical reaction whereby whole antibodies are conjugated to particles. In this example, Au nanoparticles both with and without dye are encapsulated with the DEC221 mixture of lipids with the addition of PEG-modified lipids (mPEG-DSPE), and carboxy-PEG-modified lipids (COOH-PEG-DSPE). The COOH-PEG-DSPE is reacted with antibodies using the common 1-Ethyl-3-[3-dimethylaminopropyl]carbodiimide (EDC) and N-hydroxysulfosuccinimide (Sulfo-NHS) chemistry.

As an example of potential in vitro diagnostic applications, lipid encapsulated particles both with and without MGITC dye conjugated by this method to anti-CD19 antibodies were applied to patient-derived CLL cells, which are known to express CD19 cell surface markers (FIG. 11). In this example, particles were encapsulated with DEC221, mPEG-DSPE, COOH-PEG-DSPE, and one of DPPA or DPPG (structures shown in row (A)). Darkfield micrographs (row B) clearly show labeling of the cells with gold nanoparticles. The particles scatter light intensely and appear green, red, and gold in the images. Control samples where the cells were first treated with anti-CD19 antibodies to block the CD19 receptors on cells before applying particles, show significantly less particle-binding, which indicates a specific interaction between the conjugated particles and the cells (row (C). Control samples where cells were exposed to unconjugated particles show nearly no particle binding (row D). A SERS spectrum of Anti-CD19 conjugated MGITC particles illustrates how cells bearing the targeted SERS particles can be detected by the SERS spectrum of MGITC (row E).

As a further example of in vitro diagnostic application, AuNPs encapsulated with DEC221 and mPEG-DSPE using MGITC as the dye, were targeted to CLL cells via antibody fragments (Fab). The method of Fab conjugation is schematically illustrated in FIG. 12A: Whole anti-CD20 antibodies are digested using the enzyme Ficin to produce F(ab′)₂ fragments that contain the two epitope binding regions of the antibody and cysteine (a thiol-containing amino acid) which can be exploited to bind fragments to lipids. Specifically, cysteine is known to bind to maleimide groups, thus F(ab′) fragments were reacted to lipids with headgroups modified with maleimide-PEG (mal-PEG-DSPE). The resulting lipid-anchored antibody fragment (Fab-PEG-DSPE) was inserted into the lipid layer surrounding the particles by incubating encapsulated particles with Fab-PEG-DSPE. FIG. 12B is a darkfield micrograph of anti-CD20 Fab-conjugated lipid-encapsulated AuNPs attached to patient-sourced CLL cells, which demonstrates dense labeling of cells by these particles. FIG. 12C is a darkfield micrograph of unconjugated (mPEG-DSPE/DEC221) lipid-encapsulated AuNP applied to CLL cells, which demonstrates minimal non-specific labeling of the cells. FIG. 12D shows the SERS spectrum of cell samples treated with Fab-targeted particles (black line) and unconjugated particles (grey dashed line) demonstrating SERS detection of labeled cells.

Given the foregoing results, the skilled person would be capable of practising the invention in its various aspects, including variations as described below.

By “aqueous” is meant a solution in which the liquid phase contains water, when preparing a phospholipid(s) or lipid(s) component when preparing a SERS complex of the invention. The solution can contain other solvents that are miscible with water as long as a single liquid phase is maintained. Such other solvents include the lower (C1-C6) alcohols, particularly a lower alcohol such as ethanol. It is thought, that the maximum amount of other solvent(s) that might be included, however, is limited to about 50% (v/v) of the liquid phase.

Nanoparticles of the invention can be composed of materials other than gold nanoparticles. The nanoparticles are metal nanoparticles and may be gold, silver, copper, nickel, palladium, platinum, ruthenium, rhodium, osmium, iridium, or an alloy of any of the foregoing metals. In vivo applications require that the SERS moiety to be biocompatible, and in such applications, the preferred metal would likely be gold, but might also be an alloy of gold and silver, or gold and platinum, or core-shell structures of two metals where the shell metal is gold. It is possible that a silver particle with a gold coating would be suitable.

In terms of size, nanoparticles suitable for the invention are somewhere between 2 nm to about 1000 nm, but more likely greater than 5 nm and less than 900 nm. Preferably, nanoparticles are between about 5 nm and about 300 nm, or between about 5 nm and about 100 nm. It thought that the most likely to be preferred diameter is between about 20 nm and about 90 nm.

Nanoparticles used in the foregoing experiments were generally spherical, and this shape, or at least one very similar to it, is the one would typically use when one wants to ensure that the nanoparticle is more or less fully encapsulated.

Phospholipids are a class of compounds known to those skilled in the art. One type of phospholipid is glycerol-based and includes a headgroup which can be, for example, phosphtidyl glycerol (PG), phosphotidyl choline (PC), phosphatidic acid (PA), phophatidyl ethanolamine (PE), phosphatidyl serine (PS), phosphatidyl inositol (PI), and derivitized/functionalized forms of these such as rhodamine-PE or PEG-PE. The tail groups can be comprised of hydrogen, or an acyl chain such as palmitoyl, myristyol, oleoyl, stearoyl etc. The tail group can consist of two identical groups or two different groups. For example DOPC has two oleoyl acyl chains and a phosphochoine headgroup joined by a glycerol backbone. DOPG has the same acyl chains but a phosphatidylglycerol headgroup. POPC has one palmitoyl and one oleoyl acyl chains and a phosphatidylcholine head group.

Also used in examples described herein was sphingomyelin, a sphingophospholipid. Sphingolipids include sphingosine and its derivatives, including sphingomyelin.

Phospholipids, which include sphingophosoplipids, are known to occur naturally. Included within this definition are other non-naturally occurring phospholipids that are structurally related to certain of the naturally occurring compounds in that they are amphipathic and can form layers for encapsulation of metal nanoparticles. Within the definition of phospholipids are those containing phosphtidyl glycerol (PG), phosphotidyl choline (PC), phosphatidic acid (PA), phophatidyl ethanolamine (PE), phosphatidyl serine (PS), and phosphatidyl inositol (PI) in their headgroup and joined via a glycerol moiety to one or two fatty acyl chains. Each chain can have from 6 up to 24 carbons, and may be branched or unbranched, although they are typically unbranched. Each chain can include one or more double bonds or one or more triple bonds. Chains having 12 to 24 carbon atoms are more preferred.

In a preferred embodiment, the phospholipid component of a SERS complex of the invention is a bilayer.

Examples of particular phospholipids of the invention include dioleoylphosphatiylcholine (DOPC), dipalmitoylphosphatidylcholine (DPPC), dipalmitoyl phosphatidyl glycerol (DPPG), dipalmitoyl phosphatidic acid (DPPA), distearoyl phosphatidyl ethanolamine (DSPE), dimyristoylphosphatidyl choline (DMPC), diacyl phosphatidyl glycerols, such as dimyristoyl phosphatidyl glycerol (DMPG), dipalmitoyl phosphatidyl glycerol (DPPG), and distearoyl phosphatidyl glycerol (DSPG), diacyl phosphatidyl cholines, such as dimyristoyl phosphatidylcholine (DMPC), dipalmitoyl phosphatidylcholine (DPPC), and distearoyl phosphatidylcholine (DSPC); diacyl phosphatidic acids, such as dimyristoyl phosphatidic acid (DPMA), dipalmitoyl phosphatidic acid (DPPA), and distearoyl phosphatidic acid (DSPA); and diacyl phosphatidyl ethanolamines such as dimyristoyl phosphatidyl ethanolamine (DMPE), dipalmitoyl phosphatidyl ethanolamine (DPPE) and distearoyl phosphatidyl ethanolamine (DSPE), distearoylphosphatidylethanolamine-polyethyleneglycol (DSPE-PEG), dipalmitoylphosphatidylethanolamine-polyethyleneglycol (DPPE-PEG), dioleoylphosphatidylethanolamine-polyethyleneglycol (DOPE-PEG), wherein the PEG chain can range from 200 Da to 10000 Da molecular weight, but more likely greater than 350 Da and less than 5000 Da. Preferably, PEG chain lengths would be in the range of 1 kDa to 5 kDa molecular weight. Additionally, while one end of the PEG chain is joined to the lipid species, the other end of the PEG chain may be functionalized by other groups such as a methyl group, carboxylic acid group, N-hydroxysuccinimide group, maleimide groups, cyanur group, etc, to impart chemical functionality to the outer surface of the encapsulated particle, or to facilitate further chemical modification of the PEG chain, for instance to attach a targeting species.

Sphingolipids are lipid molecules with a backbone consisting of a sphingoid base (itself containing an acyl chain) to which is bound a fatty acyl chain and a headgroup. The fatty acyl chain can contain 6-24 carbons, and may be branched or unbranched. This chain can contain 1 or more double bonds and/or one or more triple bonds and be cis or trans isomers. Chains of 12 to 24 carbons are preferred. Headgroups can, for example, be hydrogen yielding ceramides, or phosphocholine yielding sphingomyelin. A preferred embodiment of the invention includes sphingomyelin.

Preferably, a SERS complex of the invention includes mixtures of phospholipids and/or sphingolipids and/or sterols whereby the mechanical and chemical properties of the coatings can be controlled by varying the relative quantities of components in the mixture.

Bilayers are formed spontaneously based on the relative volume fraction of the headgroup and the acyl chains. Amphiphiles with single acyl chains have a cone-like shape that facilitates structures that have high curvature such as micelles, while lipids with symmetric or nearly symmetric acyl chains have more cylindrical profiles that are more conducive to forming low curvature structures such as planar supported bilayers, and vesicles.

A SERS reporter of the invention, sometimes referred to as a Raman active molecule or Raman dye refers to a molecule which exhibits a characteristic Raman spectrum upon excitation with light. Here, due to the proximity of the Raman active molecule to the surface of a metal nanoparticle within a complex of the invention, the Raman signal is enhanced, making the complex a Surface Enhanced Raman Scattering (SERS) complex. SERS reporters described in the examples disclosed herein include malachite green isothocyanate (MGITC), ethyl violet (EV), and 4-fluorobenzenethiol (4-FBT), rhodamine lissamine DSPE (Rho-PE), and L-tryptophan (Trp). Other examples of SERS reporters that can be incorporated into a SERS complex of the invention include isothiocyanate dyes such as substituted rhodamine isothicyanate (XRITC), Tetramethylrhodamine isothiocyanate, Fluorescein isothiocyanate (FITC), etc; common dyes such as Rhodamine, Texas Red, Cy3, Cy5, Cy5.5, etc; Triphenylmethane-based dyes such as Basic Fuchin, Methyl Violet, Crystal Violet, Phenol Red, Malachite Green, etc; Infrared dyes such as Indocyanine Green (ICG), Bengal Rose, etc; Thiolated aromatic compounds such as mercaptobenzoic acid, 3,5-dichlorobenzenethiol (3,5-DCT), 4-Nitrobenzenthiol (4-NBT), etc; Oxazine dyes such as phenoxazine, Nile Blue, etc. Many other SERS reporters are known, as described, for example, in US 2011/0151586, published Jun. 23, 2011, the contents of which document are incorporated herein by reference.

A SERS complex of the invention can include an additional ligand, one that has the ability to recognize and bind to partner of the ligand. A SERS complex having such a ligand can thus bind to the ligand partner. An example of such a ligand, one illustrated by results described herein, is an antibody (SERS complex ligand) which binds to an antigen (target).

In the results illustrated by laboratory examples carried out and described herein, the ligand was incorporated into an already prepared SERS complex. In one example, the SERS complex was prepared with a phosholipid containing a functional group to which the ligand could be covalently linked. The ligand was then linked to the functional group of the phospholipid in the encapsulating layer of the SERS complex. In another example, the ligand was linked to a phosholipid that was subsequently incorporated into the encapsulating layer of an existing SERS complex. Both approaches successfully produced SERS complexes having ligands that were able to recognize their targets. The skilled person will recognize here that an anchor covalently linked to the ligand is not necessarily a phospholipid but that it has to be e.g., a lipid compatible with the encapsulating layer of the SERS complex so that it can stably incorporated into the layer.

Various types of moieties that can act as a ligand when covalently linked to a component of the encapsulating layer of the SERS complex are a nucleotide, a nucleic acid molecule, a DNA molecule, an RNA molecule, an aptamer, a peptide, a protein, an amino acid, a lipid, a carbohydrate, a drug, a drug precursor, a drug candidate molecule, a drug metabolite, a vitamin, a synthetic polymer, a receptor ligand, a metabolite, an immunoglobulin, a fragment of an immunoglobulin, a domain antibody, a monoclonal antibody, a VH domain, a VL domain, a single chain antibody, a nanobody, a unibody, a monobody, an affibody, a DARPin, an anticalin, a ¹⁰Fn3 domain, a versabody, a Fab fragment, a Fab′ fragment, an Fd fragment, an Fv fragment, an F(ab′)₂ fragment, and an Fc fragment, a proteinaceous binding molecule having an antibody-like function, a glubody, a protein based on the ankyrin scaffold or the crystalline scaffold, an AdNectin, a tetranectin, an avimer, a peptoid, or a cell surface marker.

A ligand can alternatively be defined in terms of the moiety or target with which it binds or which it selectively captures. A ligand can thus be one that selectively binds to a cell, a virus, bacteria, a spore, a toxin, a protein, a peptide, and amino acid, an antigen, a lipid, a nucleic acid, a polynucleotide, an oligonucleotide, a drug, or an explosive.

The skilled person will appreciate that e.g., an antibody can be linked to the SERS complex so as to target an antigen, in one context, while in another context, an antigen might be linked to the SERS complex as a ligand which targets an antibody.

Phospholipids of the encapsulating layer of a SERS complex of the invention are amphipathic. Hydrophilic portions of phosholipids are situated towards the exterior of the layer and a ligand is linked so that it can effectively recognize its partner. In one of the examples described herein, COOH-PEG-DSPE (DSPE=distearoyl phosphatidylethanolamine) was incorporated into the encapsulating phospholipid layer during production of the SERS complex. The carboxyl group was then activated and covalently coupled to an antibody in a conventional manner. In another example, a F(ab′)₂ fragment was generated and covalently linked to maleimide-PEG-DSPE through the thiol group of the cysteine residue of the fragment. The F(ab)-PEG-DSPE was then incubated with a SERS complex to be incorporated into its encapsulating layer. The skilled person thus appreciates that the ligand is linked in these cases to the outwardly located lipid headgroup and oriented outwardly of the phospholipid layer of the SERS complex to permit binding with the target of the ligand.

In another aspect, the invention is a multiplex assay comprising two or more SERS complexes. A multiplex assay is a technique used to detect and/or quantify two or more molecules wherein the molecules are different from each other and these molecules can be detected and/or quantified in a single mixture. Multiple SERS complexes can thus be used as probes which can separately label multiple analytes. An example would be attaching different antibodies, each one of which antibodies has specific affinity for a distinctive type of cancer cell. A SERS mapping would reveal the localization of these distinctive types of cancer cells.

As used herein, the terms “about” and “approximately, when used in conjunction with ranges of dimensions of particles, compositions of mixtures or other physical properties or characteristics, is meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present invention.

The entire disclosures of all applications, patents and publications cited herein are hereby incorporated by reference. This invention may also be said broadly to be composed of the parts, elements and features referred to or indicated herein, individually or collectively, in their various possible combinations.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

### REFERENCES

(1) Loo, C.; Lowery, A.; Halas, N.; West, J.; Drezek, R. Immunotargeted Nanoshells for Integrated Cancer Imaging and Therapy. Nano Lett. 2005, 5, 709-711.
(2) Huang, X.; EI-Sayed, I. H.; Qian, W.; EI-Sayed, M. A. Cancer Cell Imaging and Photothermal Therapy in the Near-Infrared Region by Using Gold Nanorods. J. Am. Chem. Soc. 2006, 128, 2115-2120.
(3) Giljohann, D. A.; Seferos, D. S.; Daniel, W. L.; Massich, M. D.; Patel, P. C.; Mirkin, C. A. Gold Nanoparticles for Biology and Medicine. Angew. Chem. Int. Ed. 2010, NA-NA.
(4) Zhang, C.; Yeh, H.; Kuroki, M. T.; Wang, T. Single-quantum-dot-based DNA nanosensor. Nat Mater 2005, 4, 826-831.
(5) Oyelere, A. K.; Chen, P. C.; Huang, X.; EI-Sayed, I. H.; EI-Sayed, M. A. Peptide-Conjugated Gold Nanorods for Nuclear Targeting. Bioconjugate Chem. 2007, 18, 1490-1497.
(6) Kumar, S.; Harrison, N.; Richards-Kortum, R.; Sokolov, K. Plasmonic Nanosensors for Imaging Intracellular Biomarkers in Live Cells. Nano Lett. 2007, 7, 1338-1343.
(7) Qian, X.; Peng, X.; Ansari, D. O.; Yin-Goen, Q.; Chen, G. Z.; Shin, D. M.; Yang, L.; Young, A. N.; Wang, M. D.; Nie, S. In vivo tumor targeting and spectroscopic detection with surface-enhanced Raman nanoparticle tags. Nat Biotech 2008, 26, 83-90.
(8) Sha, M. Y.; Xu, H.; Natan, M. J.; Cromer, R. Surface-Enhanced Raman Scattering Tags for Rapid and Homogeneous Detection of Circulating Tumor Cells in the Presence of Human Whole Blood. J. Am. Chem. Soc 2008, 130, 17214-17215.
(9) Kneipp, J.; Kneipp, H.; Rajadurai, A.; Redmond, R. W.; Kneipp, K. Optical probing and imaging of live cells using SERS labels. J. Raman Spectrosc. 2009, 40, 1-5.
(10) Nguyen, C. T.; Nguyen, J. T.; Rutledge, S.; Zhang, J.; Wang, C.; Walker, G. C. Detection of chronic lymphocytic leukemia cell surface markers using surface enhanced Raman scattering gold nanoparticles. Cancer Lett. 2010, 292, 91-97.
(11) Hofheinz, R.; Gnad-Vogt, S. U.; Beyer, U.; Hochhaus, A. Liposomal encapsulated anti-cancer drugs. Anti-Cancer Drugs 2005, 16, 691-707.
(12) Wu, J.; Liu, Q.; Lee, R. J. A folate receptor-targeted liposomal formulation for paclitaxel. Int. J. Pharm. 2006, 316, 148-153.
(13) Torchilin, V. P. Recent advances with liposomes as pharmaceutical carriers. Nat Rev Drug Discov 2005, 4, 145-160.
(14) He, P.; Urban, M. W. Phospholipid-Stabilized Au-Nanoparticles. Biomacromolecules 2005, 6, 1224-1225.
(15) Chen, W. C.; Completo, G. C.; Sigal, D. S.; Crocker, P. R.; Saven, A.; Paulson, J. C. In vivo targeting of B-cell lymphoma with glycan ligands of CD22. Blood 2010, 115, 4778-4786.
(16) Park, J. W. Tumor targeting using anti-her2 immunoliposomes. J. Controlled Release 74, 95-113.
(17) Allen, T. M.; Moase, E. H. Therapeutic opportunities for targeted liposomal drug delivery. Adv. Drug Delivery Rev. 1996, 21, 117-133.
(18) Park, J. W.; Hong, K.; Kirpotin, D. B.; Colbern, G.; Shalaby, R.; Baselga, J.; Shao, Y.; Nielsen, U. B.; Marks, J. D.; Moore, D.; Papahadjopoulos, D.; Benz, C. C. Anti-HER2 Immunoliposomes. Clin. Cancer Res. 2002, 8, 1172-1181.
(19) Thaxton, C. S.; Daniel, W. L.; Giljohann, D. A.; Thomas, A. D.; Mirkin, C. A. Templated Spherical High Density Lipoprotein Nanoparticles. J. Am. Chem. Soc. 2009, 131, 1384-1385.
(20) Cormode, D. P.; Skajaa, T.; van Schooneveld, M. M.; Koole, R.; Jarzyna, P.; Lobatto, M. E.; Calcagno, C.; Barazza, A.; Gordon, R. E.; Zanzonico, P.; Fisher, E. A.; Fayad, Z. A.; Mulder, W. J. M. Nanocrystal Core High-Density Lipoproteins: A Multimodality Contrast Agent Platform. Nano Lett. 2008, 8, 3715-3723.
(21) Bakshi, M. S.; Possmayer, F.; Petersen, N. O. Role of Different Phospholipids in the Synthesis of Pearl-Necklace-Type GoldSilver Bimetallic Nanoparticles as Bioconjugate Materials. J. Phys. Chem. C 2007, 111, 14113-14124.
(22) Bakshi, M. S.; Kaur, G.; Thakur, P.; Banipal, T. S.; Possmayer, F.; Petersen, N. O. Surfactant Selective Synthesis of Gold Nanowires by Using a DPPC—Surfactant Mixture as a Capping Agent at Ambient Conditions. J. Phys. Chem. C 2007, 111, 5932-5940.
(23) Chuang, C.; Chen, Y. Raman scattering of L-tryptophan enhanced by surface plasmon of silver nanoparticles: vibrational assignment and structural determination. J. Raman Spectrosc. 2009, 40, 150-156.
(24) Aliaga, A. E.; Osorio-Román, I.; Leyton, P.; Garrido, C.; Cárcamo, J.; Caniulef, C.; Célis, F.; Díaz F., G.; Clavijo, E.; Gómez-Jeria, J. S.; Campos-Vallette, M. M. Surface-enhanced Raman scattering study of L-tryptophan. J. Raman Spectrosc. 2009, 40, 164-169.
(25) Sullen, R. M. A.; Li, J. K.; Zou, S. Direct Correlation of Structures and Nanomechanical Properties of Multicomponent Lipid Bilayers. Langmuir 2009, 25, 7471-7477.
(26) Lapinski, M. M.; Castro-Forero, A.; Greiner, A. J.; Ofoli, R. Y.; Blanchard, G. J. Comparison of Liposomes Formed by Sonication and Extrusion: Rotational and Translational Diffusion of an Embedded Chromophore. Langmuir 2007, 23, 11677-11683.
(27) Maulucci, G.; De Spirito, M.; Arcovito, G.; Boffi, F.; Castellano, A. C.; Briganti, G. Particle Size Distribution in DMPC Vesicles Solutions Undergoing Different Sonication Times. Biophys. J. 2005, 88, 3545-3550.
(28) Lueck, H. B.; Daniel, D. C.; McHale, J. L. Resonance Raman study of solvent effects on a series of triarylmethane dyes. J. Raman Spectrosc. 24, 363-370.
(29) Rule, K. L.; Vikesland, P. J. Surface-Enhanced Resonance Raman Spectroscopy for the Rapid Detection of Cryptosporidium parvum and Giardia lamblia. Environmental Science & Technology 2009, 43, 1147-1152.
(30) Pettinger, B.; Krischer, K. Comparison of cross-sections for absorption and surface-enhanced resonance Raman scattering for rhodamine 6G at coagulated silver colloids. J. Electron Spectrosc. Relat. Phenom. 1987, 45, 133-142.
(31) Michaels, A. M.; Nirmal, M.; Brus, L. E. Surface Enhanced Raman Spectroscopy of Individual Rhodamine 6G Molecules on Large Ag Nanocrystals. J. Am. Chem. Soc. 1999, 121, 9932-9939.
(32) Pieczonka, N. P. W.; Aroca, R. F. Single molecule analysis by surfaced-enhanced Raman scattering. Chem. Soc. Rev. 2008, 37, 946.
(33) Zeidel, M. L.; Ambudkar, S. V.; Smith, B. L.; Agre, P. Reconstitution of functional water channels in liposomes containing purified red cell CHIP28 protein. Biochemistry 1992, 31, 7436-7440.
(34) Richter, R. P.; Maury, N.; Brisson, A. R. On the Effect of the Solid Support on the Interleaflet Distribution of Lipids in Supported Lipid Bilayers. Langmuir 2005, 21, 299-304.
(35) Rossetti, F. F.; Textor, M.; Reviakine, I. Asymmetric Distribution of Phosphatidyl Serine in Supported Phospholipid Bilayers on Titanium Dioxide. Langmuir 2006, 22, 3467-3473.
(36) Richter, R. P.; Brisson, A. R. Following the Formation of Supported Lipid Bilayers on Mica: A Study Combining AFM, QCM-D, and Ellipsometry. Biophys. J. 2005, 88, 3422-3433.
(37) Kasbauer, M.; Junglas, M.; Bayerl, T. Effect of Cationic Lipids in the Formation of Asymmetries in Supported Bilayers. Biophys. J. 1999, 76, 2600-2605.
(38) Jensen, L.; Schatz, G. C. Resonance Raman Scattering of Rhodamine 6G as Calculated Using Time-Dependent Density Functional Theory. J. Phys. Chem. A 2006, 110, 5973-5977.
(39) Zhang, J.; Li, X.; Sun, X.; Li, Y. Surface Enhanced Raman Scattering Effects of Silver Colloids with Different Shapes. J. Phys. Chem. B 2005, 109, 12544-12548.
(40) Chen, J.; Jiang, J.; Gao, X.; Gong, J.; Shen, G.; Yu, R. Gold-aggregated, dye-embedded, polymer-protected nanoparticles (GDPNs): A new type of tags for detection with SERS. Colloids Surf., A 2007, 294, 80-85.
(41) Machatha, S. G.; Yalkowsky, S. H. Estimation of the ethanol/water solubility profile from the octanol/water partition coefficient. Int. J. Pharm. 2004, 286, 111-115.
(42) Blaser, G.; Sanderson, J. M.; Wilson, M. R. Free-energy relationships for the interactions of tryptophan with phosphocholines. Org. Biomol. Chem. 2009, 7, 5119-5128.
(43) Ross, M.; Steinem, C.; Galla, H.; Janshoff, A. Visualization of Chemical and Physical Properties of Calcium-Induced Domains in DPPC/DPPS LangmuirBlodgett Layers. Langmuir 2001, 17, 2437-2445.
(44) Richter, R. P.; Bérat, R.; Brisson, A. R. Formation of solid-supported lipid bilayers: an integrated view. Langmuir 2006, 22, 3497-3505.

1-21. (canceled)

22. The method of claim 23, wherein said nanoparticles of step (a) are conjugated to monoclonal antibodies and/or other targeting ligands using one or both of physical and chemical means.

23. A method of preparing metal nanoparticles for use in surface enhanced Raman scattering (SERS), the method comprising the steps of:
(a) mixing metal nanoparticles and at least one SERS reporter with an aqueous solution comprising phospholipid alone, sphingolipid alone, phospholipid and sphingolipid, phospholipid and sterol, sphingolipid and sterol, or phospholipid and sphingolipid and sterol, to form a mixture; and
(b) agitating the mixture to induce encapsulation of the nanoparticles and SERS reporter by a layer comprising said phospholipid alone, sphingolipid alone, phospholipid and sphingolipid, phospholipid and sterol, sphingolipid and sterol, or phospholipid and sphingolipid and sterol.

24. The method of claim 23, wherein the said phospholipid alone, sphingolipid alone, phospholipid and sphingolipid, phospholipid and sterol, sphingolipid and sterol, or phospholipid and sphingolipid and sterol is selected such that said layer is a bilayer.

25. The method of claim 24, wherein the layer at least partially encapsulates said nanoparticles and SERS reporter in step (b).

26. The method of claim 24, wherein the layer fully encapsulates said nanoparticles and SERS reporter in step (b).

27. The method of claim 24, wherein agitating the mixture comprises sonicating the mixture.

28. (canceled)

29. (canceled)

30. The method according to claim 28 wherein said phospholipids are one type or a mixture of phospholipids, said sphingolipids are one type or a mixture of sphingolipids, and said sterols are one type or a mixture of sterols.

31. (canceled)

32. The method according to 30, wherein the aqueous solution of step (a) comprise a glycerophospholipid, and wherein the glycerophospholipid is optionally a diacylglycerophospholipid.

33. (canceled)

34. The method of claim 32, wherein the dycerophospholipids of step (a) comprises a phosphosphingolipid.

35. (canceled)

36. The method of claim 27, wherein said phospholipids, sphingolipids and sterols have headgroups that are positively charged, negatively charged, zwitterionic, or neutral, and wherein optionally said phospholipids and sphingolipids have headgroups selected from the group consisting of phosphatidyl choline (PC), phosphatidyl ethanolamine (PA), phosphatidyl inositol (PI), and phosphatidyl glycerol (PG).

37. (canceled)

38. (canceled)

39. The method claim 27, wherein step (a) includes first mixing the nanoparticles with SERS reporter molecules and stirring to facilitate adsorption of the SERS reporter molecules on an outer surface of the nanoparticles, thereafter mixing the nanoparticles with SERS reporter molecules adsorbed thereto with the phospholipids to form said mixture.

40. The method of claim 27, wherein step (a) includes mixing said phospholipid alone, sphingolipid alone, phospholipid and sphingolipid, phospholipid and sterol, sphingolipid and sterol, or phospholipid and sphingolipid and sterol having SERS reporter molecules covalently bound thereto with other said phospholipid alone, sphingolipid alone, phospholipid and sphingolipid, phospholipid and sterol, sphingolipid and sterol, or phospholipid and sphingolipid and sterol, and thereafter mixing the nanoparticles with said phospholipid alone, sphingolipid alone, phospholipid and sphingolipid, phospholipid and sterol, sphingolipid and sterol, or phospholipid and sphingolipid and sterol having said SERS reporter molecules bound thereto to form said mixture.

41. The method of claim 27, wherein step (a) includes first dissolving the SERS reporter molecules into the solution and thereafter mixing therewith the nanoparticles to form the mixture.

42. The method of claim 27, wherein the SERS reporter is a hydrophobic organic dye and the dye is incorporated into the encapsulation layer during step (b).

43. (canceled)

44. (canceled)

45. The method of claim 27, including controlling a distribution of charged lipids in the encapsulation layer by addition of acids and/or salts in said solution.

46. The method of claim 27, further comprising the step of: (c) separating encapsulated nanoparticles formed in step (b) from unbound phospholipid alone, sphingolipid alone, phospholipid and sphingolipid, phospholipid and sterol, sphingolipid and sterol, or phospholipid and sphingolipid and sterol and SERS reporter molecules by centrifugation.

47. (canceled)

48. The method of claim 27, wherein a ligand is covalently linked to a said phospholipid alone, sphingolipid alone, phospholipid and sphingolipid, phospholipid and sterol, sphingolipid and sterol, or phospholipid and sphingolipid encapsulating a nanoparticle obtained in step (b), wherein the ligand is optionally selected from the group consisting of a nucleic acid molecule, a DNA molecule, an RNA molecule, an aptamer, a peptide, a protein, an amino acid, a lipid, a carbohydrate, a drug, a drug precursor, a drug candidate molecule, a drug metabolite, a vitamin, a synthetic polymer, a receptor ligand, a metabolite, an immunoglobulin, a fragment of an immunoglobulin, a domain antibody, an antibody, a monoclonal antibody, a VH domain, a VL domain, a single chain antibody, a nanobody, a unibody, a monobody, an affibody, a DARPin, an anticalin, a ¹⁰Fn3 domain, a versabody, a Fab fragment, a Fab′ fragment, an Fd fragment, an Fv fragment, an F(ab′)₂ fragment, and an Fc fragment, a proteinaceous binding molecule having an antibody-like function, a glubody, a protein based on the ankyrin scaffold or the crystalline scaffold, an AdNectin, a tetranectin, an avimer, a peptoid, or a cell surface marker.

49. (canceled)

50. (canceled)

51. The method of claim 27, further comprising mixing encapsulated nanoparticles produced in step (b) with a lipid having a ligand covalently thereto, and agitating the mixture to incorporate the lipid having a ligand covalently thereto into the encapsulation layer of the encapsulated nanoparticles or incubating the mixture to incorporate the lipid having a ligand covalently thereto into the encapsulation layer of the encapsulated nanoparticles.

52. (canceled)

53. The method of claim 61, wherein the phospholipids of step (a) include a phospholipid having a functional group covalently linked to its headgroup for subsequent linkage to a ligand.

54. The method of claim 53, wherein the functional group is a maleimide group, carboxyl group or a protected carboxyl group, wherein the maleimide group, carboxyl group or protected carboxyl group is optionally covalently linked to the headgroup by a polyethylene glycol.

55. (canceled)

56. (canceled)

57. (canceled)

58. (canceled)

59. The method of claim 27, wherein the nanoparticles are of a metal selected from the group consisting of gold, silver, copper, nickel, palladium, platinum, ruthenium, rhodium, osmium, iridium, and alloys of any of the foregoing metals, wherein said nanoparticles are optionally capped by positively or negatively charged ligands with any and all combinations of phospholipids.

60. (canceled)

61. The method of claim 27, wherein the aqueous solution of step (a) comprises phospholipids having one or more hydrocarbon chain(s) being any one or combination of saturated, monounsaturated, and polyunsaturated, and optionally comprising one or more of:
dioleoylphosphatiylcholine (DOPC), dipalmitoylphosphatidylcholine (DPPC), dipalmitoyl phosphatidyl glycerol (DPPG), dipalmitoyl phosphatidic acid (DPPA), distearoyl phosphatidyl ethanolamine (DSPE), dimyristoylphosphatidyl choline (DMPC), a diacyl phosphatidyl glycerol, dimyristoyl phosphatidyl glycerol (DMPG), dipalmitoyl phosphatidyl glycerol (DPPG), a distearoyl phosphatidyl glycerol (DSPG), a diacyl phosphatidyl choline, distearoyl phosphatidylcholine (DSPC); a diacyl phosphatidic acid, such as dimyristoyl phosphatidic acid (DPMA), distearoyl phosphatidic acid (DSPA), a diacyl phosphatidyl ethanolamine, dimyristoyl phosphatidyl ethanolamine (DMPE), dipalmitoyl phosphatidyl ethanolamine (DPPE), distearoylphosphatidylethanolamine-polyethyleneglycol (DSPE-PEG), dipalmitoylphosphatidylethanolamine-polyethyleneglycol (DPPE-PEG), dioleoylphosphatidylethanolamine-polyethyleneglycol (DOPE-PEG), wherein the PEG chain is from 200 Da to 10000 Da.

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. A surface enhanced Raman scattering (SERS) complex comprising a metal nanoparticle and a SERS reporter encapsulated by a phospholipid layer, wherein the SERS reporter is covalently linked to a molecule of the phospholipid layer, the SERS reporter is a hydrophobic organic dye incorporated into the phospholipid layer, or is physisorbed or covalently linked to the nanoparticle.

78. The complex of 77, further comprising a ligand covalently linked to a phospholipid of the phospholipid layer.

79. (canceled)

80. The complex of claim 78, wherein the ligand is covalently linked to the headgroup of the phospholipid, and the ligand is selected from the group consisting of a nucleic acid molecule, a DNA molecule, an RNA molecule, an aptamer, a peptide, a protein, an amino acid, a lipid, a carbohydrate, a drug, a drug precursor, a drug candidate molecule, a drug metabolite, a vitamin, a synthetic polymer, a receptor ligand, a metabolite, an immunoglobulin, a fragment of an immunoglobulin, a domain antibody, a monoclonal antibody, a VH domain, a VL domain, a single chain antibody, a nanobody, a unibody, a monobody, an affibody, a DARPin, an anticalin, a ¹⁰Fn3 domain, a versabody, a Fab fragment, a Fab′ fragment, an Fd fragment, an Fv fragment, an F(ab′)₂ fragment, and an Fc fragment, a proteinaceous binding molecule having an antibody-like function, a glubody, a protein based on the ankyrin scaffold or the crystalline scaffold, an AdNectin, a tetranectin, an avimer, a peptoid, or a cell surface marker.

81. The complex of claim 78, wherein the ligand is an antibody or an antibody fragment.

82. (canceled)
